(19) United States
(12) Patent Application Publication
(10) Pub. No.: US 2011/0091482 A1
(43) Pub. Date: Apr. 21, 2011

(54) EXPRESSION OF KIR IN HUMAN CANCER CELLS AS A BIOMARKER FOR IMMUNO-ESCAPE AND CANCER METASTASIS

(75) Inventors: Daniel C. Chan, Aurora, CO (US); Di Zheng, Aurora, CO (US); Zhiyong Zhang, Aurora, CO (US)

(73) Assignee: The Regents of the University of Colorado, A Body Corporate, Denver, CO (US)

(21) Appl. No.: 12/936,892

(22) PCT Filed: Apr. 9, 2009
(86) PCT No.: PCT/US09/40066

§ 371 Date: Dec. 29, 2010

Related U.S. Application Data
(60) Provisional application No. 61/044,335, filed on Apr. 11, 2008.

(51) Int. Cl. C12Q 1/68; C12Q 1/02; C40B 30/00; G01N 33/574; A61K 39/395; A61K 31/713; A61K 31/7088; A61K 38/02; A61P 35/00

(52) U.S. Cl. 424/173.1; 435/29; 506/7; 435/6; 435/7.23; 514/44 A; 514/44 R; 514/19.3

(57) ABSTRACT

The present invention is based on the finding that the level of KIR expression in a cancer cell is significant with regard to tumor invasion and metastasis. Therefore, the present invention concerns methods and compositions for evaluating cancer in a patient based on KIR protein or mRNA expression. The invention also provides methods and compositions for treating cancer using a KIR inhibitor and methods of screening for KIR inhibitors.

This application claims benefit of priority to U.S. Provisional Application Ser. No. 61/044,335, filed Apr. 11, 2008, the entire contents of which are hereby incorporated by reference.

This invention was made with government support under Grant Nos. P30 CA044634, P30CA046937, P50 CA058187 awarded by the National Institutes of Health-National Cancer Institute. The government has certain rights in the invention.

### BACKGROUND OF THE INVENTION

I. Field of the Invention

The present invention relates generally to the fields of genetics, biochemistry, immunology, molecular biology, and oncology. In certain aspects the invention is related to prognosis and therapy for cancer. More particularly, it concerns NK cells, KIR proteins and KIR nucleic acids, inhibitors of KIR, and their relevance to immuno-escape and cancer metastasis.

II. Description of Related Art

Cancer is a multi-step process consisting of multiple genetic events and mutations. Genetic alterations associated with cancer progression and metastasis are further complicated by tumor-cell interactions with surrounding tissues, especially host immune cells within the tumor microenvironment (Kopfstein and Christofori, 2006; Lorusso and Ruegg, 2008). The process of tumor metastasis is highly selective and consists of a series of sequential, inter-related steps which include invasion through extracellular matrix, intravasation, survival in the circulation, extravasation into a distant site, and progressive growth at that site.

Invasion and metastasis of cancer cells to distant lymph nodes usually indicates advanced stages of malignancy. Metastasis is the main cause of death among these advanced staged cancer patients. Lymph nodes are part of the important organs for immunodefense. Thus, it is ironic that cancer cells select lymph nodes and lymph channels for its spreading and invasion (metastasis). How cancer cells escape immuno-surveillance and immune destruction have been gaining attention recently as a component of a more general process of cancer immunoediting. This process describes the concepts and pathways that lead from cancer immunosurveillance to tumor escape as a result of three processes: elimination, equilibrium and escape (Dunn et al., 2002).

The concept of tumor immuno-surveillance suggests that the natural immune system can detect tumor antigens that result from genetic alterations, thereby preventing the development of cancers (Waldhauer and Steinle, 2008; Swann and Smyth, 2007; Kopfstein and Christofori, 2006; Lorusso and Ruegg, 2008; Albini et al., 2008). However, since cancers nevertheless emerge and progress, this suggests that the immune system has failed to eradicate completely the progressed cancer cells, possibly due to acquired immune tolerance (Ostrand-Rosenberg, 2008; Zitvogel et al., 2006). The newly evolved concept, immuno-editing, suggests that the immune system not only protects the host against development of primary nonviral cancer but also inadvertently sculpts tumor immunogenicity for increasingly aggressive growth and further resistance to immune destruction as they progress (Dunn et al., 2002; Smyth et al., 2006; Reiman et al., 2007).

Therefore there is a need to identify factors, such as genes or family of genes, that are involved in immuno-escape, cancer invasion and metastatic processes, for prognostic and therapeutic benefits.

### SUMMARY OF THE INVENTION

The present invention is based on the finding that the natural killer (NK) cell immunoglobulin-like receptors (KIR) are expressed in metastatic human lung cancer cells, and correlate with aggressive metastatic behavior. Therefore, the present invention concerns methods and compositions for evaluating cancer in a patient based on KIR expression in cancer cells. The invention also provides methods and compositions for treating cancer using a KIR inhibitor and methods of screening for KIR inhibitors.

In certain embodiments, the invention provides a method of evaluating cancer in a patient comprising determining the level of KIR in a biological sample containing cancer cells obtained from the patient, wherein an elevated level of KIR on the cancer cells as compared to a control is indicative of an aggressive metastatic form of cancer and/or a poor prognosis.

Biological samples include, but are not limited to, tissue and serum samples. In some embodiments, a tissue sample is obtained from a biopsy, such as a biopsy of tissue that may be cancerous or tumorigenic or a metastasis. In other embodiments, the sample is obtained from a biopsy of tissue from a lymph node. In other embodiments, the sample is a serum sample, a pleural fluid sample, a peritoneal fluid sample, a spinal fluid sample, a bronchoalveolar lavage fluid sample, a cerebral spinal fluid sample, a pleural effusion sample, or a peritoneal effusion sample.

In certain embodiments, the control is a sample from a non-cancerous subject or from non-cancerous tissue. In other embodiments, the control is from non-metastasized cancerous tissue. In some embodiments, the level of KIR in the control is a known level of KIR. In other embodiments, the level of KIR in the control is evaluated prior to, simultaneously with, or after the level of KIR in the sample is evaluated. In particular embodiments, the level of KIR to be detected is located on the cancer cells, and is distinguished from those expressed on the NK cells that may infiltrate inside the tumor tissues or tumor microenvironment. One of skill in the art will be aware of various methods to distinguish KIR detected on cancer cells from those expressed on the NK cells, including but not limited to dual immunofluoresence, FACS, and GFP. In particular embodiments, to differentiate KIR-expressed cancer cells from KIR-expressed NK, NKT or T cells, various bi- or multi-markers may be used, including but not limited to (KIR+CD56); (KIR+CD56+CD3); (KIR+CD56+CD16); (KIR+CD56+NKp46); (KIR+CD56+NKp30); (KIR+CD56+NKp44); (KIR+NK1.1). In certain aspects, these markers in combination with one or more markers from Table 1 may be used to differentiate KIR expressed on cancer cells from KIR expressed on immunce cells. These markers allow differentiation between cancer cells expressing KIR from those of NK, NKT and T cells. It has been found that except KIR, non of these markers are expressed on the human cancer cells, or expressed only in a very low level (almost undetectable).

It is contemplated that “KIR expression” may refer to mRNA expression or to protein expression. In certain embodiments, the level of KIR mRNA is evaluated, measured, and/or determined. This may be done using any method by which mRNA expression levels are evaluated, measured, or determined. A variety of such methods are well known to those of skill in the art, and these include, but are not limited to, those involving complementary probes or primers, amplification primers, cDNAs, etc. Such methods may involve RT-PCR, in situ hybridization (ISH), and/or arrays or biochips for evaluating RNA expression. In other embodiments, the level of KIR protein is evaluated, measured, and/or determined. A variety of such methods are well known to those of skill in the art, and these include, but are not limited to, immunohistochemistry or immunocytochemistry. Such methods may involve arrays or biochips for evaluating protein expression. It is anticipated that virtually any test for analysis of KIR expression may be used with the present invention, for example, ELISA, immunoassay, radioimmunoassay (RIA), immunoradiometric assay, fluoroimmunoassay, chemiluminescent assay, bioluminescent assay, gel electrophoresis, Western blot analysis or in situ hybridization assay.

In some cases, the KIR level is higher than the level of a control by virtue of expression being detected at any level. For example, KIR protein level may be higher than the control by virtue of its being detected at any level when the sample is analyzed by immunohistochemistry, where the control shows no detectable level when analyzed by immunohistochemistry. However, in other embodiments, the level of KIR is at least about or at most about 110, 120, 130, 140, 150, 160, 170, 180, 190, 200, 210, 220, 230, 240, 250, 260, 270, 280, 290, 300% or more, or any range derivable therein, compared to the level in a control. In other embodiments, the level of KIR expression is at least about or at most about 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-fold or times, or any range derivable therein, greater than the level of a control. The level of expression refers to the level of detectable expression by any particular method of measuring or determining that level.

In methods of the invention, a patient may also be treated or more aggressively treated for cancer after the level of KIR (protein and/or mRNA) is evaluated in the patient if the level is indicative of an aggressive form of cancer. Methods may also involve further or previous tests for cancer. Consequently, methods may also involve treating the patient with a conventional cancer treatment. In certain embodiments, the treatment is chemotherapy, radiotherapy, surgery, gene therapy, hormonal therapy, and/or immunotherapy.

In some embodiments, the patient may exhibit symptoms of cancer, is at risk for cancer, or has been diagnosed with cancer. The invention may be used in connection with any cancer that expresses KIR, including but not limited to oral cancer, oropharyngeal cancer, nasopharyngeal cancer, respiratory cancer, a urogenital cancer, a gastrointestinal cancer, a central or peripheral nervous system tissue cancer, an endocrine or neuroendocrine cancer, a hematopoietic cancer, a glioma, a sarcoma, a carcinoma, a lymphoma, a melanoma, a fibroma, a meningioma, brain cancer, oropharyngeal cancer, nasopharyngeal cancer, renal cancer, biliary cancer, prostatic cancer, pheochromocytoma, pancreatic islet cell cancer, a Li-Fraumeni tumor, thyroid cancer, parathyroid cancer, pituitary tumors, adrenal gland tumors, osteogenic sarcoma tumors, multiple neuroendrcine type I and type II tumors, breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, rectal cancer or skin cancer. In particular embodiments, the level of KIR to be detected is located on these cancer cells and is distinguished from those expressed on the NK cells that may infiltrate in the tumor tissues. The KIR expressed on cancer cells may be differentiated from KIR expressed on NK cells by various methods known to those of skill in the art, including but not limited to dual immunofluoresence by FACS analysis and GFP.

In further embodiments of the invention, a biological sample is obtained from a patient. In other embodiments of the method, the entity evaluating the sample for KIR levels did not directly obtain the sample from the patient. Therefore, methods of the invention involve obtaining the sample indirectly or directly from the patient. To achieve these methods, a doctor, medical practitioner, or their staff may obtain a biological sample for evaluation. The sample may be analyzed by the practitioner or their staff, or it may be sent to an outside or independent laboratory. The test may provide information regarding a quantitative level of KIR, or the test may indicate directly or indirectly that the test was positive or negative for KIR. In the case of KIR protein, it is specifically contemplated that the evaluation may indicate simply that a sample is positive or negative for KIR protein. In some embodiments, the invention further comprises reporting the level of KIR expression in the sample and/or the control. In particular embodiments, the level of KIR to be detected is located on these cancer cells and is distinguished from those expressed on the NK cells that may infiltrate in the tumor tissues. The KIR expressed on cancer cells may be differentiated from KIR expressed on NK cells by various methods known to those of skill in the art, including but not limited to dual immunofluoresence, FACS, and GFP.

In other embodiments, treatment may involve administering to the patient a KIR inhibitor. A KIR inhibitor refers to a substance that specifically inhibits KIR's immuno-tolerance and renders it sensitive to NK cell killing either by suppressing KIR expression on cancer cells or disrupting KIR structures that prevents its recognition as “self” by the NK cells. This may be done separately or in conjunction with the other cancer treatments. In certain embodiments, the KIR inhibitor inhibits KIR activity, while in others the KIR inhibitor inhibits KIR expression. In particular embodiments, KIR activity on cancer cell is the resistance to cytolytic killing by NK cells. The KIR inhibitor may be a nucleic acid, a peptide, an antibody, or a small molecule. In particular embodiments, the KIR inhibitor is a KIR siRNA that inhibits KIR expression. In other embodiments, the KIR inhibitor is a peptide or an antibody that binds with KIR on cancer cells and renders it sensitive to cytolytic killing by NK cells.

Methods of the invention specifically include methods of treating cancer comprising administering to a patient an effective amount of a KIR inhibitor. The term “effective amount” refers to the amount needed to inhibit KIR and effects a therapeutic benefit relevant to cancer. The term “therapeutic benefit” refers to anything that promotes or enhances the well-being of the subject with respect to cancer. A list of nonexhaustive examples of this includes extension of the subject's life by any period of time, decrease or delay in the neoplastic development of the disease, decrease in hyperproliferation, reduction in tumor growth, delay of metastases, reduction in cancer cell or tumor cell proliferation rate, and a decrease in pain to the subject that can be attributed to the subject's condition.

The KIR inhibitor or other cancer treatment may be administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctival, intravesicularlly, mucosally, intrapericardially, intraumbilically, orally, by inhalation, by injection, by infusion, by continuous infusion, by localized perfusion bathing target cells directly, via a catheter, or via a lavage.

Other embodiments of the invention concern methods of screening for a KIR inhibitor comprising (a) contacting KIR with a candidate substance; and (b) assaying the level of KIR, wherein a reduction in KIR indicates that the candidate substance is a candidate KIR inhibitor. KIR activity encompasses binding MHC class 1 molecules and inhibiting signals from activating receptors by a negative or inhibitory signal. In some embodiments of the invention, a KIR inhibitor is an agent that mimics MHC class 1 molecules. In other embodiments, a KIR inhibitor is one that blocks the activity or function of KIR. Assays for evaluating this activity are contemplated as part of the invention. Methods may involve evaluating KIR activity in the presence and absence of the candidate inhibitor to determine whether the candidate is an inhibitor. The candidate inhibitor may be a nucleic acid, a peptide, an antibody, or a small molecule. In particular embodiments, the KIR inhibitor is a peptide or an antibody. In other embodiments, the KIR inhibitor is a KIR siRNA that inhibits KIR expression. It is contemplated that such methods may be performed using high throughput screening, arrays, or a biochip such that multiple candidates can be evaluated. In vitro cytolytic killing assays by NK cells on various cancer cells with or without expression of KIR, in the absence or presence of KIR inhibitors is used to determine the activity of KIR inhibitor effects.

In further embodiments, the invention provides a method of monitoring treatment of cancer in a patient comprising (a) determining the level of KIR in a first sample from the patient; (b) determining the level of KIR in a second sample from the patient after treatment is effected; and (c) comparing the level of KIR in the first sample with the level of KIR in the second sample to assess a change and monitor treatment.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-F: Orthotopic H2122-GFP tumors in athymic nude rats: Adenocarcinoma H2122-GFP tagged cells were implanted in the left of nude rats via intra-tracheal instillation. (FIG. 1A) Primary tumors in left lung of animal. (FIG. 1B) Primary tumor in left lung, micrometastases in right lung and one large metastase in mediastinal lymph node. (FIG. 1C) Metastases found in spleen (arrow). (FIG. 1D) Micrometastases in pancreas (arrow). (FIG. 1E) micrometastase in lumbar lymph node (arrow) and (FIG. 2F) micrometastases in kidneys and adrenal gland.

FIGS. 2A-D: Metastasis of KIR positive H2122-PL-GFP cells re-implanted orthotopically in the left lung of nude mouse. (FIG. 2A) Nude mouse implanted with H2122-PL-GFP. (FIG. 2B) Metastases in lung, liver, spleen, pancreas, guts, abdominal lymph vessels, and paws. (FIG. 2C) Metastases in skull, eyes, and nose. (FIG. 2D) Metastases in bone marrows in legs.

FIGS. 3A-D: Re-establishment of cell cultures from human lung tumors implanted subcutaneously or orthotopcially in nude rats and re-isolated from these nude rat several weeks after tumor established. (FIG. 3A) Cells grown from subcutaneous tumor (SQ). (FIG. 3B) Cells grown form primary lung tumors (PL). (FIG. 3C) Cells grown from right lung metastases (ML). (FIG. 3D) Cells grown from distant metastases such as lumbar lymph nodes (DM).

FIG. 4A-B: Gene signals of adhesion molecules in H2122 (FIG. 4A) Integrins/Invasion. (FIG. 4B) Integrin α and β subunits/invasion.

FIG. 5: MMP genes in H2122G FP cell lines MMP (proteolytic proteins); involved in basement membrane invasion.

FIG. 6: Chemokines R/ligand genes in H2122GFP liners; provide for growth in new microenvironment.

FIG. 7: Genes for growth factors in H2122GFP lines; provide for growth in new microenvironment.

FIG. 8: Genes for Angiogenesis in H2122GFP lines; provide for growth in new microenvironment.

FIGS. 9A-B: KIR gene signals in H2122-GFP parental cells and in H2122 tumor cells re-established from orthotopic nude rats: PL (primary tumor in left lung), ML (metastase in right lung) and DM (distant metastases in pancreas). (FIG. 9A) Gene signals for KIR family versus H2122 tumors. (FIG. 9B) Normalized gene signals for KIR family versus H2122 tumors.

FIGS. 10A-D: Flow Cytometric analysis of KIR proteins in H2122-GFP parental and tumor cells re-established from orthotpic nude rats, using anti-KIR 2DL1 antibodies conjugated with APC. (FIG. 10A) Less than 2% double positive (GFP and APC) cells were detected in H2122-GFP parental cells. (FIG. 10B) 15% double positive KIR cells in PL primary tumors in left lung. (FIG. 10C) 30% double positive KIR cells in ML metastases from right lung. (FIG. 10D) 60% double positive KIR cells in DM distant metastases from pancreas.

FIGS. 11A-D: Flow cytometric analysis of percentage of KIR positive cells in four human cancer cell lines and tumors passaged in orthotopic rodents, using antibodies against KIR subtypes 2DL1, 2DL2, 2DS4 and 3DL1. (FIG. 11A) Orthotopic A549 adenocarcinoma re-established from PL, ML and DM (mediastinal lymph node). (FIG. 11B) Orthotopic Mia-PaCa pancreatic carcinoma re-established from PL implanted in pancreas of nude mouse, DM distant metastases from abdominal lymph node and DM from ascites. (FIG. 11C) Melanoma WM115 re-established from PL tumors implanted in left lung of nude rat, ML metastases from right lung, DM distant metastase from liver and another DM distant metastases from abdominal lymph node. (FIG. 11D) Breast carcinoma McF-7 re-established from PL primary tumor implanted in left lung of nude rat and ML metastases in mediastinal lymph node.

FIGS. 12A-B: Anti-proliferative effects of human and rodent NK cells on H2122-GFP parental cell lines and H2122 metastatic subclones (K3) with high KIR expression, measured by fluorescence plate reader. (FIG. 12A) H2122-GFP parental cells and K3 subclones were incubated with various amount of NK cells, indicated as Effector (NK)/Target (cancer) ratio. After incubation for 5 days, cell proliferation was measured through GFP fluorescence intensity in a 96-well plate reader and normalized with control cells. Effective inhibition was compared by IC50 inhibition, based on ETR. (FIG. 12B) Similar experiments were carried out with rodent NK cells.

FIG. 13: KIR expression in H2122-GFP parental cells after co-cultured with various amount of human NK cells. Resistant cells were selected from H2122-GFP that survived from coculturing with human NK cells and stained with anti-KIR-2DL1 antibodies and analyzed with flow cytometry.

FIGS. 14A-L: Immunohistochemistry of anti-KIR 2DL1 antibodies in tissue microarray consisting of human lung cancers with matched lymph node metastases. Patients with grade IV SCLC (FIGS. 14A, B) and lymph node metastases (FIGS. 14B, D). Patients with grade III adenocarcinomas (FIGS. 14E, G) and lymph node metastases (FIGS. 14F, H). Patients with grade III squamous cell carcinomas (FIGS. 141, K) and lymph node metastases (FIGS. 14J, L).

FIGS. 15A-F: Anti-KIR 2DL1 immuno-reactivity in H2122-GFP parental cells and subclones PL.PL (K3) with high expression of KIR. Single cell suspension of H2122 parental cells and K3 subclones were grown on chamber slides for 24 hours and processed for immuno-staining with anti-KIR 2DL1 antibodies similar to standard ihc. Negative controls, parental cells (FIG. 15A) and K3 subclones (FIG. 15B) were reacted without primary antibodies. H2122 parental cells (FIGS. 15C,D) and K3 subclones (FIGS. 15E,F) were reacted with anti-KIR 2DL1 antibodies at 1:100 dilution. Strong anti-KIR immunoreactivities are detected in K3 subclones, especially between the cell junctions (arrows).

FIGS. 16A-E: Immunohistochemistry of anti-KIR immunoreactivities in tissue microarray consisting of human multi-tumors. Normal Lung (FIG. 16A1), adenocarcinoma (FIG. 16A2), squmamous cell carcinoma (FIG. 16A3), SCLC (FIG. 16A4), reactive lymph nodes (FIG. 16B1), Hodgkin's lymphoma (FIG. 16B2), Non-Hodgkin's B cell lymphoma (FIG. 16B3), Non-Hodgkin T cell lymphoma (FIG. 16B4), normal ovary (FIG. 16C1), ovarian adenocarcinoma (FIG. 16C2), normal uterus endometrium (FIG. 16D1), uterus adenocarcinoma (FIG. 16D2), normal skin (FIG. 16E1), and melanoma (FIG. 16E2).

### DESCRIPTION OF ILLUSTRATIVE EMBODIMENTS

As discussed herein, the present invention is based on the finding that the level of KIR expression in a cancer cell is significant with regard to tumor invasion and metastasis. In particular, a whole family of killer cell immunoglobulin-like receptors (KIR) receptors which were expressed in GFP-tagged human lung cancer cells re-isolated from primary tumors implanted in the left lung of nude rats has been unexpectedly detected. These KIRs increased progressively with distance of metastases from primary tumors. In contrast, KIRs were very low or not expressed at all in the parental lung cancer cell line in vitro. Immuno-fluorescent flow cytometry demonstrated that KIR proteins are also expressed in H2122 GFP-tagged metastatic lung cancer cells after xeno-transplantation in athymic nude rats. Furthermore, NK immunoglobulin-like inhibitor receptors (KIR) are also aberrantly expressed on the surface of human lung cancer cells after orthotopic implantation in the lungs of athymic nude rats. Thus, metastatic cancer cells with high KIR expression levels are more resistant than parental cells to cytolytic killing by human or rodent NK cells in vitro. Continuous co-culture of parental lung cancer cells with cytolytic human or rodent NK cells results in the selection of resistant cancer cell subpopulations with significantly elevated expression of surface KIRs.

It is theorized that certain cancer cells aberrantly express KIR receptors due to interaction with the NK cells, which allows these cancer cells to “fool” the immune surveillance system as “self” and therefore allows escape through the lymph channels and invasion to distant sites as micrometastases. Additional features besides the capability of immuno-escape are needed by these cells for invasion and distant metastasis.

Using immunohistochemical analysis in human tissue-microarrays, expression of KIR proteins was detected on cancer cells in multiple human malignant tumors of advanced stages, suggesting that aberrant KIR expression is quite common among human aggressive tumors and metastases. Therefore, the presence of KIRs in tumor cells reveals new frontiers in cancer biology, cancer metastasis and cancer immunology, and may lead to an improved understanding of the mechanisms of metastasis due to escape from immunosurveillance. The expression of KIR proteins in human cancer cells will serve as important biomarkers for metastatic cancers. It is therefore an embodiment of the present invention to provide new cancer treatment and anti-metastatic strategies.

### I. KIR AND NK CELLS

Natural killer (NK) cells, T cells and B cells comprise the three subsets of lymphocytes in mammalian immune systems. NK cells, which play an important role in the innate and adaptive immune system, are large granular lymphocytes capable of killing tumors, viruses and virally infected cells. NK cells participate in the immune system by producing cytokines to stimulate other cells of the immune system and in the direct destruction of infections or transformed cancer cells. NK cells are fundamental in defenses against viruses, primarily herpes viruses. Upon activation, NK cells release cytokines and chemokines that induce inflammatory response, modulate hematopoiesis, control monocyte and granulocyte cell growth and function and influence adaptive responses. Under normal conditions, NK cells are mostly confined to peripheral blood, spleen and bone marrow, but can migrate to inflamed tissues in response to different chemo-attractants.

The functions of NK cells are regulated by a balance between activating receptors and inhibitory receptors that interact with their ligand such as MHC class I or MHC class I-related molecules (non-classical MHC class I) on the target cells (Rajaram et al., 1990). Natural killer (NK) cells also play a role in the control of tumor growth in vivo and prevent the rapid dissemination of metastatic tumors (Carrega et al., 2008). They are capable of recognizing and eliminating a majority of malignant cancer cells and provide a first line of defense against tumor development (Waldhauer and Steinle, 2008; Swan and Smyth, 2007). The infiltration of NK cells have been reported in almost all human malignancies including breast (Georgiannos et al., 2003), colorectal (Coca et al., 1997), esophageal (Hsia et al., 2005), gastric (Ishigami et al., 2000), hepatocellular (Taketomi et al, 1998), laryngeal (Gonzalez et al., 1998), lung (Carrega et al., 2008), lymphoma (Dukers et al., 2001), melanoma (Azogui et al., 1991), ovarian (Belisle et al., 2007) prostate (Tarle et al., 1993), renal (Schleypen et al., 2006) and uterine cervix (Vaguer et al., 1990). Despite strong immune-surveillance, a subpopulation of aggressive cancer cells nevertheless traverses lymphatic and vascular vessels and metastasizes to lymph nodes and distant organs.

One such group of inhibitory receptors are killer cell Ig-like receptors (KIR). KIRs are members of the immunoglobulin superfamily of receptors and are encoded on human chromosome 19q13.4 as part of the chromosome region designated the leukocyte receptor cluster (LRC) (Uhrberg et al., 1997; Parham, 2005). In humans, 14 KIR genes are clustered in a 150 kb region of human chromosome 19 called the leukocyte receptor complex (LRC). KIR genes are characterized by the number of Ig domains (2D or 3D) and by the length of their cytoplasmic tail. Long-tailed KIRs (2DL or 3DL) contain immuno-receptor tyrosine-based inhibition motifs (ITIMs), which recruit the phosphatase SHP-1 upon receptor engagement and induce inhibitory signals. Short-tailed KIRs (2DS or 3DS) lack IT1Ms and send activating signals to NK cells by association with the adaptor signaling molecule DAP12 via a charged amino acid in the transmembrane region. Rodents lack KIRs and instead use structurally distinct Ly49 CTLD receptors to bind MHC class 1 molecules on target cells. Using Affymetrix analysis and immuno-fluorescent flow cytometry, the inventors have found that only the KIRs are elevated and differentially expressed on the metastastic cancer cells while all the down stream signaling molecules such as ITIMs, SHP-1 or DAP12 are barely detectable in these cancer cells. The flow cytometric studies further confirmed that most of the KIR proteins are membrane bound and exposed extracellularly. The data suggests and demonstrates that KIR expressed cancer cells are resistant to cytolytic killing by NK or NKT cells.

NK cells are mediated by the integration of inhibitory (KIR) and activating (NCR) signals sent by the cell surface receptors upon binding to ligands present in target cells. NK cells recognize major histocompatibility complex (MHC) class 1 molecules in normal cells via surface KIR receptors that deliver signals that suppress, rather than activate NK cell function. NK cells do not kill normal cells due to the presence of MHC class 1 ligand, which are recognized by the inhibitory receptor KIR that inhibits signals from activating receptors. Thus, the KIR inhibitory receptors on NK cells discriminate healthy from transformed cells by surveying the surface expression of MHC class 1 molecules, and the NK cells kill those cancer cells that have lost or express insufficient amounts of MHC class 1. The KIR subtype that activates NK cells has two extracellular Ig domains with a short intra-cytoplamic tail (KIR2DS), and the other KIR subtypes that inhibit NK cells have two or three extracellular Ig domains with a long intra-cytoplasmic tail (KIR2DL, KIR3DL). Normally, the expression of KIR receptors and its activation or inhibition of NK functions is unique to NK cells.

On the other hand, altered or absent MHC class 1 in transformed cancer cells fail to bind with KIR and therefore cannot stimulate a negative or inhibitory signal, resulting in the activating receptor signals that trigger the NK cells to release granule contents, leading to induction of apoptosis in the targeted cell. The expression of NK cell receptors on cancer cells may help them escape from normal immune surveillance as they may fool the immune system as “self” thereby allowing the cancer cells to invade through the immune system such as lymph channels and lymph nodes and escaping to distant organs as metastases.

A. Functional Aspects

When the present application refers to the function or activity of KIR expressed on cancer cells, it is meant that the molecule in question has the ability to inhibit cytolytic killing by NK cell. Determination of which molecules possess this activity may be achieved using assays familiar to those of skill in the art. For example, transfer of genes encoding KIR or variants thereof, into cancer cells that have a functional KIR product will identify, by virtue of an increased level of resistance to cytolytic killing by NK cells, those molecules having a KIR inhibitor function. An endogenous KIR polypeptide refers to the polypeptide encoded by the cell's genomic DNA.

On the other hand, when the present invention refers to the function or activity of a KIR inhibitor, one of ordinary skill in the art would further understand that this includes, for example, the ability to specifically or competitively bind KIR or an ability to reduce or inhibit its activity, such as reduce its ability to activate the killing by NK cell. Thus, it is specifically contemplated that a KIR modulator may be a molecule that affects KIR expression, such as by binding a KIR-encoding transcript. Determination of which molecules are suitable modulators of KIR may be achieved using assays familiar to those of skill in the art.

In vitro cytolytic killing assays by NK cells are used to determine the effects of expression of KIRs in cancers, especially those metastatic cancer cells. It is hypothesized that certain level of KIR expression on cancer cells will make these cells resistant to killing by NK cells. In the presence of certain effective KIR inhibitors as defined here, resistance of killing by NK cells can be reversed.

### II. KIR DETECTION METHODS

It is within the general scope of the present invention to provide methods for the detection of proteins and mRNA. Any method of detection known to one of skill in the art falls within the general scope of the present invention.

A. Protein Detection

In certain embodiments, the present invention concerns determining the expression level of the protein KIR in cancer cells. In particular, the level of KIR to be detected is expressed on the cancer cells and is differentiated from KIR expressed on the NK cells that may infiltrate inside the tumor tissues or tumor microenvironment. A variety of markers may be used to differentiate KIR expressed on cancer cells from KIR expressed on immune cells. Table 1 identifies non-limiting examples of such markers. These markers should allow one of ordinary skill in the art to differentiate cancer cells expressing KIR from those of NK, NKT and T cells. Except KIR, none of these markers are expressed on the human cancer cells, or expressed only at a very low, almost undetectable level.

TABLE 1
Cell surface markers of natural killer (NK) cells

| Marker | Molecule/function | Distribution |
|---|---|---|
| Relatively | | |
| NK specific | | |
| NK1.1 | NKR-P1 | ~All NK cells, some T cells |
| DX5 (in rodents) | CD49b | ~All NK cells, some T cells |
| CD16 | Fc receptor | Most NK cells, some T cells |
| CD94 (KLRD1) | Pairs with | ~All NK cells (different |
| | NKG2A/C/E | levels), some T cells |
| NKG2A (KLRC1) | Inhibitory receptor | Subset of NK cells, some |
| | | T cells |
| Ly49 (KLRA) | Inhibitory/ | NK cells subsets, some |
| (in rodents) | stimulatory receptors | T cells |
| KIR (in humans) | Inhibitory/ | NK cells subsets, some |
| | stimulatory receptors | T cells |
| NKp46 | Stimulatory receptor | All NK cells (different levels) |
| NKp30 | Stimulatory receptor | All NK cells (different levels) |
| NKp44 | Stimulatory receptor | All NK cells (different levels) |
| Other | | |
| NKG2D | Stimulatory/ | ~All NK cells, activated |
| | costimulatory | CD8⁺ T cells, activated |
| | receptor | macrophages |
| CD2 | Immune modulation | All NK cells, all T cells |
| CD5 | Immnue modulation | All NK cells, all T cells |
| CD11a | LFA-1a | NK and T cells, granulocytes, |
| | | macrophages |
| CD11b | Mac-1a | NK cells, granulocytes, |
| | | macrophages |
| CD11c | p150a | NK, T, and B cells; |
| | | granulocytes; macrophages |
| CD18 | b2 integrin | All leukocytes |
| CD45 | B220 | All leukocytes |
| CD56 ( in humans) | | NK cells, some T cells |
| CD57(in humans) | | NK cells, some T cells |
| 2B4 | | All NK cells, some T cells |
| KLRG1 (MAFA) | | NK subset, some T cells, |
| | | possibly mast cells |
| gp49 | | NK cells, mast cells |
| IL-2/IL-15Rb | Component of IL-2/ | NK cells, T cells |
| | IL15 receptor | |

Fc, crystallizablc fragment;
IL, interleukin;
LFA, leukocyte function-associated antigen;
Mac, macrophage.

As used herein, a “protein,” “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the proteinaceous composition may comprise at least one antibody, for example, a KIR. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).
1. FACS

One method that may be employed to differentiate between KIR on the cancer cell and KIR expressed on the NK cells that may infiltrate the tumor tissues involves the use of fluorescence-activated cell sorting (FACS). FACS is a specialised type of flow cytometry, which provides a method for sorting a heterogeneous mixture of biological cells into two or more containers, one cell at a time, based upon the specific light scattering and fluorescent characteristics of each cell. Such methods are known to those of skill in the art.
2. Fluorescence Energy Transfer (FRET)

Another method that may be employed to differentiate between KIR on the cancer cell and KIR expressed on the NK cells that may infiltrate the tumor tissues involves FRET, or fluorescence resonance energy transfer. In another exemplary embodiment, Fluorescence Resonance Energy Transfer (FRET) may be used to detect KIR and NK cells in a sample. In such binding assays, the fluorescent reporter molecules commonly have overlapping spectral properties such that the emission of a donor molecule overlaps with the excitation spectra of an acceptor molecule. The latter donor molecule is thereby excited and emits the absorbed energy as fluorescent light. In competition assay formats, the fluorescent energy of the donor molecule is either quenched by the test molecule or energy transfer between the donor and acceptor is inhibited. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects may be diminished or eliminated (U.S. Pat. No. 5,981,200).
3. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize KIR or to evaluate the amount KIR in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer. Specific molecular markers are characteristic of particular cancer types.

Visualising an antibody-antigen interaction can be accomplished in a number of ways. In the most common instance, an antibody is conjugated to an enzyme, such as peroxidase, that can catalyse a colour-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immmunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g., FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody (the secondary antibody must be against the IgG of the animal species in which the primary antibody has been raised). This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.
4. Antibodies

Another embodiment of the present invention are antibodies, in some cases, a KIR antibody. It is understood that antibodies can be used for inhibiting or modulating KIR. It is also understood that this antibody is useful for screening samples from human patients for the purpose of detecting KIR present in the samples. The antibody also may be useful in the screening of expressed DNA segments or peptides and proteins for the discovery of related antigenic sequences. In addition, the antibody may be useful in passive immunotherapy for cancer. All such uses of the said antibody and any antigens or epitopic sequences so discovered fall within the scope of the present invention.

In certain embodiments, the present invention involves antibodies. For example, all or part of a monoclonal, single-chain, or humanized antibody may function as a modulator of KIR. Other aspects of the invention involve administering antibodies as a form of treatment or as a diagnostic to identify or quantify a particular polypeptide, such as KIR. As detailed above, in addition to antibodies generated against full length proteins, antibodies also may be generated in response to smaller constructs comprising epitopic core regions, including wild-type and mutant epitopes.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (monoclonal antibodies) are recognized to have certain advantages, e.g., reproducibility and large-scale production, and their use is generally preferred. The invention thus provides monoclonal antibodies of the human, murine, monkey, rat, hamster, rabbit and even chicken origin.

The term “antibody” is used to refer to any antibody-like molecule that has an antigen binding region, and includes antibody fragments such as Fab′, Fab, F(ab′)₂, single domain antibodies (DABs), Fv, scFv (single-chain Fv), and the like. The techniques for preparing and using various antibody-based constructs and fragments are well known in the art. Means for preparing and characterizing antibodies are also well known in the art (see, e.g., Harlow and Lane, 1988; incorporated herein by reference).

The methods for generating monoclonal antibodies (monoclonal antibodies) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

Monoclonal antibodies may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonal antibodies. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat. Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.
5. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay

(ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, additional advantages may be through the use of a secondary binding ligand such as a second antibody and/or a biotin/avidin ligand binding arrangement, as known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.
6. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.
7. Protein Arrays

Protein array technology is discussed in detail in Pandey and Mann (2000) and MacBeath and Schreiber (2000), each of which is herein specifically incorporated by reference.

These arrays, typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as KIR.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

B. Nucleic Acid Detection

In addition to their use in directing the expression of KIR modulator proteins, polypeptides and/or peptides, the nucleic acid sequences disclosed herein have a variety of other uses. For example, they have utility as probes or primers for embodiments involving nucleic acid hybridization. They may be used in diagnostic or screening methods of the present invention. Detection of nucleic acids encoding KIR or KIR modulators are also encompassed by the invention. See WO 2004/048933. In certain embodiments, the present invention concerns determining the level of KIR expression by determining the level of gene expression. In other embodiments, the invention provides for an inhibitor of KIR, wherein the inhibitor may be a nucleic acid. Generally, the present invention concerns polynucleotides and oligonucleotides, isolatable from cells, that are free from total genomic DNA and that are capable of expressing all or part of a protein or polypeptide. The polynucleotides or oligonucleotides may be identical or complementary to all or part of a nucleic acid sequence encoding a KIR amino acid sequence. These nucleic acids may be used directly or indirectly to assess, evaluate, quantify, or determine KIR expression.

As used in this application, the term “KIR polynucleotide” refers to a KIR-encoding nucleic acid molecule that has been isolated essentially or substantially free of total genomic nucleic acid to permit hybridization and amplification, but is not limited to such. Therefore, a “polynucleotide encoding KIR” refers to a DNA segment that contains wild-type, mutant, or polymorphic KIR polypeptide-coding sequences isolated away from, or purified free from, total mammalian or human genomic DNA. A KIR oligonucleotide refers to a nucleic acid molecule that is complementary or identical to at least 5 contiguous nucleotides of a KIR-encoding sequence, which is the cDNA sequence encoding human KIR.

It also is contemplated that a particular polypeptide from a given species may be represented by natural variants that have slightly different nucleic acid sequences but, nonetheless, encode the same protein.

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including such sequences from KIR encoding sequences.
1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65 C, and washing in 0.1×SSC/0.1% SDS at 68 C (Ausubel et al., 1989).

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.
2. In situ Hybrization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.
3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a KIR-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.
4. Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of KIR with respect to diagnostic, as well as preventative and treatment methods of the invention.
5. Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g., up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g., covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610;287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; W00138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### III. SCREENING METHODS

In some embodiments, the present invention provides a method of screening for a KIR modulator. Compounds may be screened to find modulators that could binds to MHC class 1 molecules, or inhibit the activity or function of KIR. KIR activity encompasses binding MHC class 1 molecules and inhibiting signals from activating receptors by a negative or inhibitory signal. In one embodiment, binding affinity assays and E3 liagase enzyme activity assays may be used for determining inhibitor efficiency. One of skill in the art would be aware that there are several methods available, including but not limited to those described below.

The function of KIR expressed in cancer cells is different from that expressed in NK cells. KIR on cancer cells makes them resistant to killing by NK cells and allows them to escape from immunosurveillance and acquire the capability of metastasis, while KIR expression on NK cells allows them to bind with MHC-1 molecules that are found in most cells thereby eliciting the inhibitory signals.

A. Screening for KIR Activity

In some embodiments of the present invention, methods of assaying whether the candidate inhibits KIR activity. These methods may involve screening for the activity of KIR. KIR activity may be evaluated using any of the methods and compositions disclosed herein, including assays involving evaluating KIR's binding activity, inhibition of the activity of MHC class I molecules on KIR, or KIR's ability to inhibit apoptosis. Any other the compounds or methods described herein may be employed to implement these methods.

Assays to evaluate the level of expression of a polypeptide are well known to those of skill in the art. This can be accomplished also by assaying KIR mRNA levels, mRNA stability or turnover, as well as protein expression levels. It is further contemplated that any post-translational processing of KIR may also be evaluated, as well as whether it is being localized or regulated properly. In some cases an antibody that specifically binds KIR may be used.

Furthermore, it is contemplated that the status of the gene may be evaluated directly or indirectly, by evaluating genomic DNA sequence comprising the KIR coding regions and noncoding regions (introns, and upstream and downstream sequences) or mRNA sequence. The invention also includes determining whether any polymorphisms exist in KIR genomic sequences (coding and noncoding). Such assays may involve polynucleotide regions that are identical or complementary to KIR genomic sequences, such as primers and probes described herein.

B. Modulators of KIR

The present invention further comprises methods for identifying modulators of KIR activity. These assays may comprise random screening of large libraries of candidate substances; alternatively, the assays may be used to focus on particular classes of compounds selected with an eye towards structural attributes that are believed to make them more likely to modulate the function of KIR.

By function, it is meant that one may assay for a measurable effect on KIR activity or binding activity. To identify a KIR modulator, one generally will determine the activity or level of inhibition or modulation of KIR in the presence and absence of the candidate substance, wherein a modulator is defined as any substance that alters these characteristics. For example, a method generally comprises providing a candidate modulator; admixing the candidate modulator with an isolated protein or cell expressing the protein; measuring one or more characteristics of the protein or cell; and comparing the characteristic measured with the characteristic of the protein or cell in the absence of said candidate modulator, wherein a difference between the measured characteristics indicates that said candidate modulator is, indeed, a modulator of the protein or cell. Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

As used herein the term “candidate substance” refers to any molecule that may be a “modulator” of KIR, i.e., potentially affect KIR activity or binding activity, directly or indirectly. A modulator may be a “KIR inhibitor,” which is a compound that overall effects an inhibition of KIR activity, which may be accomplished by inhibiting KIR expression, translocation or transport, function, expression, post-translational modification, location, half-life, or more directly by preventing its activity, such as by binding MHC class 1 molecules.

The candidate substance may be a protein or fragment thereof, a small molecule, or even a nucleic acid molecule. An example of pharmacological compounds will be compounds that are structurally related to KIR, or a molecule that binds MHC class 1 molecules. In some embodiments, the crystal structure of KIR and/or a KIR protein may be used to develop small molecule inhibitors that disrupt KIR-DNA or KIR-MHC class I interactions or KIR-NK cell interactions. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single-chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor or activator according to the present invention may be one which exerts its inhibitory or activating effect upstream, downstream or directly on KIR. Regardless of the type of inhibitor or activator identified by the present screening methods, the effect of the inhibition or activator by such a compound results in alteration in KIR activity as compared to that observed in the absence of the added candidate substance.

C. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### IV. PROTEINACEOUS COMPOUNDS

A. Peptides

Inhibitors of KIR may be peptides. Peptides of the current invention will comprise molecules of 5 to no more than about 50 residues in length. A particular length may be less than 39 residues, less than 35 residues, less than 30 residues, less than 25 residues, less than 20 residues, less than 15 residues, or less than 13, including 5, 6, 7, 8, 9, 10, 11 or 12 residues, and ranges of 5-11 residues, 5-15 residues, 5-20 residues, 5-25 residues, 5-30 residues, 5-35 residues, 5-38 residues, or 5-40 residues. The peptides may be generated synthetically or by recombinant techniques, or phage display library approach, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration), as described in further detail below.

The peptides may be labeled using various molecules, such as fluorescent, chromogenic or colorimetric agents. The peptides may also be linked to other molecules, including other anti-cancer agents. The links may be direct or through distinct linker molecules. The linker molecules in turn may be subject, in vivo, to cleavage, thereby releasing the agent from the peptide. Peptides may also be rendered multimeric by linking to larger, and possibly inert, carrier molecules.

B. Synthesis

The peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

C. Variants of KIR Inhibitors

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent or improved molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below.

In making substitutional variants, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0 ±1); glutamate (+3.0 ±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure. See, for example, Johnson et al., (1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of KIR, but with altered and even improved characteristics.

D. Fusion Proteins

A specialized kind of insertional variant is the fusion protein. This molecule generally has all or a substantial portion (e.g., an intracellular, transmembrane or extracellular domain) of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

E. Purification of Proteins/Peptides

It may be desirable to purify KIR or fragments or inhibitors thereof Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.). A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### V. NUCLEIC ACID VECTORS

In particular embodiments, the inhibitor or candidate substance of the present invention may be an isolated nucleic acid or a recombinant vector the invention concerns isolated DNA segments and recombinant vectors. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a truncated KIR polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to the a particular gene, such as the human KIR gene. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides, for example, a modified gelonin toxin. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (Sambrook et al., 1989; Ausubel et al., 1996, both incorporated herein by reference). In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

A. Expression Vectors or Expression Cassettes

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.
1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. The identity of tissue-specific promoters or elements, as well as assays to characterize their activity, is well known to those of skill in the art.
2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).
3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.
4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)
5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.
6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.
7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.
8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as a modified protein-encoding sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE's COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

D. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubinstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubinstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

E. Methods of Gene Transfer

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

### VI. METHODS OF THERAPY

In some embodiments, the invention provides compositions and methods for the diagnosis and treatment of cancer. In one embodiment, the invention provides a method of treating cancer comprising administering to a patient an effective amount of an inhibitor of the interaction of KIR and KIR. This treatment may be further combined with additional cancer treatments. One of skill in the art will be aware of many treatments that may be combined with the methods of the present invention, some but not all of which are described below.

The present invention also involves, in another embodiment, the treatment of cancer. The types of cancer that may be treated, according to the present invention, is limited only by the involvement of KIR. By involvement, it is not even a requirement that KIR be mutated or abnormal—the overexpression of this tumor suppressor may actually overcome other lesions within the cell. Thus, it is contemplated that a wide variety of tumors may be treated using KIR inhibition therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Peptide Therapy

One therapy approach is the provision, to a subject, of KIR inhibitor polypeptide, fragments, synthetic peptides, mimetics or other analogs thereof. The protein/peptide may be produced by recombinant expression means or, if small enough, generated by an automated peptide synthesizer. Formulations would be selected based on the route of administration and purpose including, but not limited to, liposomal formulations and classic pharmaceutical preparations.

B. Antibody Therapy

Applicants also contemplate the use of antibodies to KIR. Antibodies will be administered according to standard protocols for passive immunotherapy. Administration protocols would generally involve intratumoral, local or regional (to the tumor) administration, as well as systemic administration.

In addition, the antibody reagent may be altered, such that it will have one or more improved properties. The antibody may be recombinant, i.e., an antibody gene cloned into an expression cassette which is then introduced into a cell in which the antibody gene was not initially created. The antibody may be single-chain, a fragment (Fab, Fv, Vh, ScFv), chimeric or humanized.

C. siRNA

The KIR inhibitor may be a KIR siRNA that inhibits KIR expression. siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically-synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25 mer lengths with the endogenous nuclease complex that converts long dsRNAs to siRNAs in vivo. They do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertrophy with inhibitors of TRP channels intermittently, such as within brief window during disease progression.

D. Formulations and Routes for Administration to Patients

In some embodiments, the invention provides a method of treating cancer comprising administering to a patient an effective amount of an inhibitor of the interaction of KIR and KIR. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

E. Cancer Combination Treatments

In some embodiments, the method further comprises treating a patient with cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis or other therapeutic intervention. It also is conceivable that more than one administration of the treatment will be desired.
1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.
2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.
3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).
4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Gene Therapy

In yet another embodiment, the secondary treatment is a gene therapy in which a therapeutic polynucleotide is administered before, after, or at the same time as a KIR inhibitor is administered. Delivery of a KIR inhibitor in conjunction with a vector encoding one of the following gene products may have a combined anti-hyperproliferative effect on target tissues. A variety of proteins are encompassed within the invention, some of which are described below.
1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.
2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1 ,} and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/KIR2, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.
3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

G. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

H. Dosage

The amount of therapeutic agent to be included in the compositions or applied in the methods set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. Thus, in this regards, the concentration of the therapeutic agent in the compositions set forth herein can be any concentration. In some particular embodiments, the total concentration of the drug is less than 10%. In more particular embodiments, the concentration of the drug is less than 5%. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Orthotopic Lung Model in Nude Rats

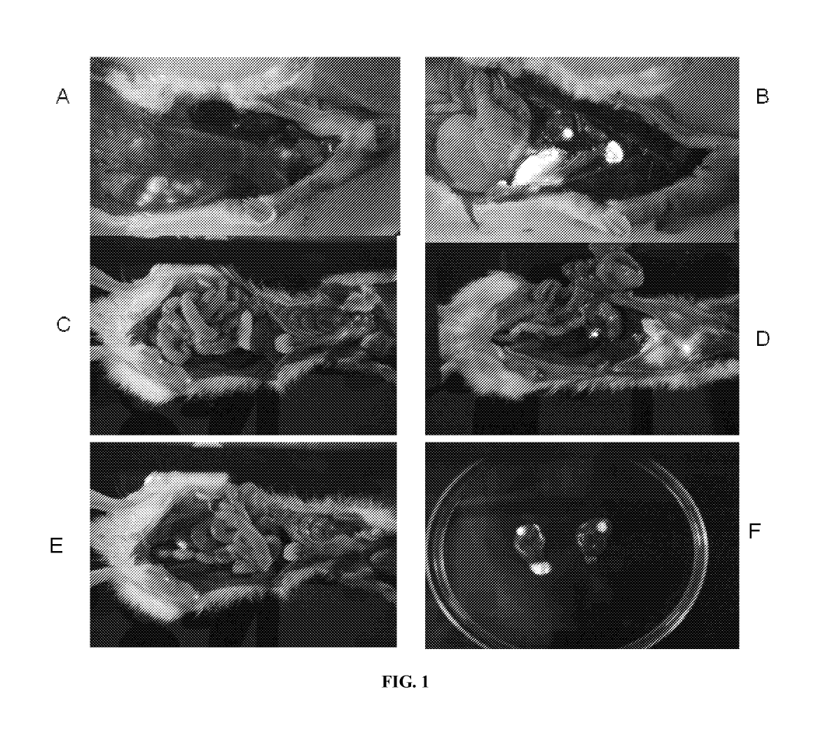

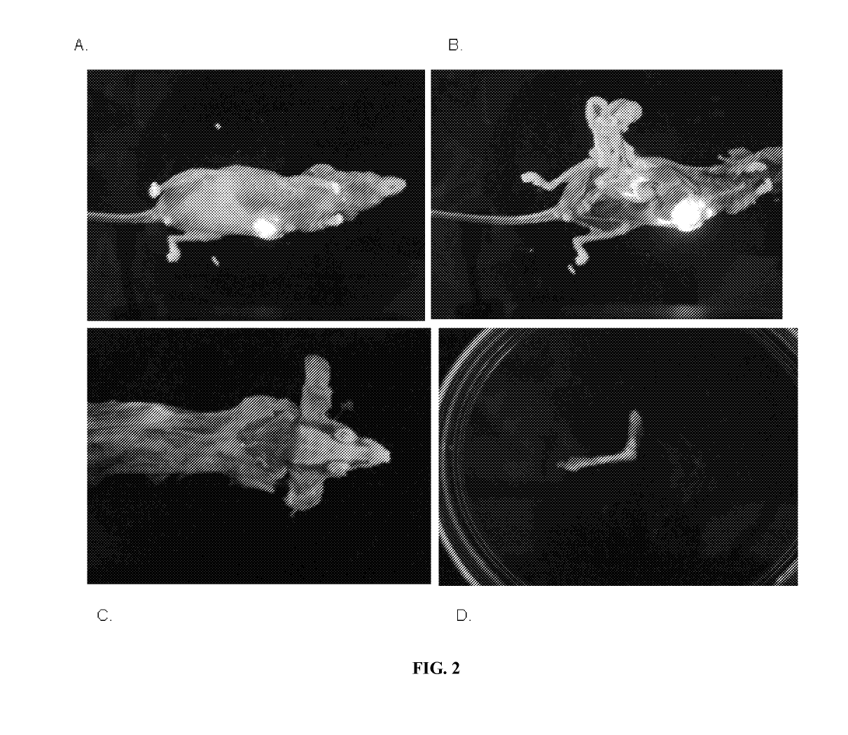

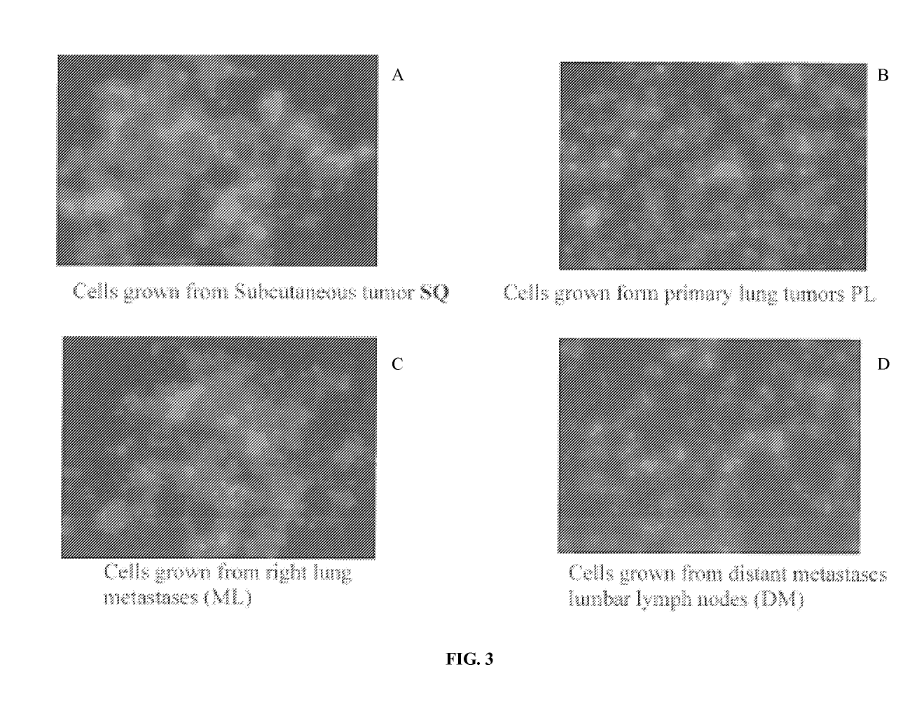

Method: H2122 (CRL-5985) is a hyperdiploid human lung adenocarcinoma (NSCLC), originally derived from metastatic pleural effusion (ATCC, Rockville, Md.). GFP-tagged H2122 was used to assess tumor growth and metastasis in athymic nude rats using a whole-body in vivo fluoresecent imaging system (Yang et al., 2001) Human lung cancer cell line H2122 adenocarcinoma was tagged with GFP (green fluorescent protein) and implanted subcutaneously in the rear flank or orthotopically in the left lung of athymic nude rats. In vivo fluorescent imaging was used to follow patterns of tumor growth and metastasis formation. When animals showed signs heavy breathing due to metastases, tumor tissues were isolated aseptically from primary sites of implantations, plus metastases in the right lung and distant metastases (FIGS. 1A-F). These tumors were mechanically disseminated and allowed to regrow again in tissue culture (FIGS. 3A-D). RNAs were isolated from these early passages cancer cells and were chipped with Affymetrix HG U133 plus 2, or re-implanted subcutaneously and orthotopically in nude rats again for further comparison studies.

Results: When implanted subcutaneously in athymic rodents, H2122 grow rapidly and aggressively into solid tumors but rarely metastasizes to distant organs. When implanted orthotopically into the left lung of athymic nude rats, H2122 grows aggressively as a primary tumor, and within two weeks metastasizes contra-laterally to the right lungs, as well as to the thymus and thoracic walls. Additionally, H2122 cells often escape the thoracic cavity and invade distant organs including pancreas, adrenal glands, spleen, liver and abdominal lymph nodes, as shown in FIGS. 1A-F. The manner of cancer spreading is very similar to what was found in human lung cancer patients. Thus, the H2122 orthotopic model in athymic nude rats provides an excellent model to follow the genetic changes that occur as tumor cells invade and interact with various metastatic microenvironments.

### Example 2

### Detection of KIR Gene Expression on H2122 Parental and Metastatic Cells

Methods: Gene and protein expression in KIR-expressed metastases isolated from different sites in the orthotopic lung model in nude rats and an equivalent melanoma models is examined in order to determine genes/proteins involved in the immuno-escape, tumor invasion and metastasis. DNA microarrays are used to analyze the metastatic cancer cells tagged with GFP and re-isolated from the orthotopic animal models. Experiments are carried out to examine protein and phosphoprotein targets of KIR, in metastatic cells from different sites of metastasis. Appropriate antibodies are used to validate the expression of the genes or proteins that are identified as of interest.

Rodent KLR receptors (Ly49) (equivalent to human KIR) are ectopically expressed in selected human lung cancer cells and determine if the resultant cell lines become more immuno-tolerant and gain metastatic features in athymic nude mice and nude rat models. The same experiment is performed in human melanoma cells, in addition to evaluating the role of plexin gene in melanoma metastasis. The role of rodent KLRs alone in immuno-tolerance and tumorigenesis is first studied. Then the combined expression of both KLR and plexin for metastasis in orthotopic lung and melanoma cancer models is tested. In vivo fluorescence imaging is used to follow tumor growth and metastasis.

Several cell lines derived from the fluorescently tagged primary tumors and from distant metastases of aseptically dissected nude rats were re-established and visualized by whole-body fluorescent imaging. Gene expression profiling of these early passaged cells were acquired by Affymetrix Hu-133 plus 2 and analyzed with the GeneSpring GX 7.3.1 (Agilent).

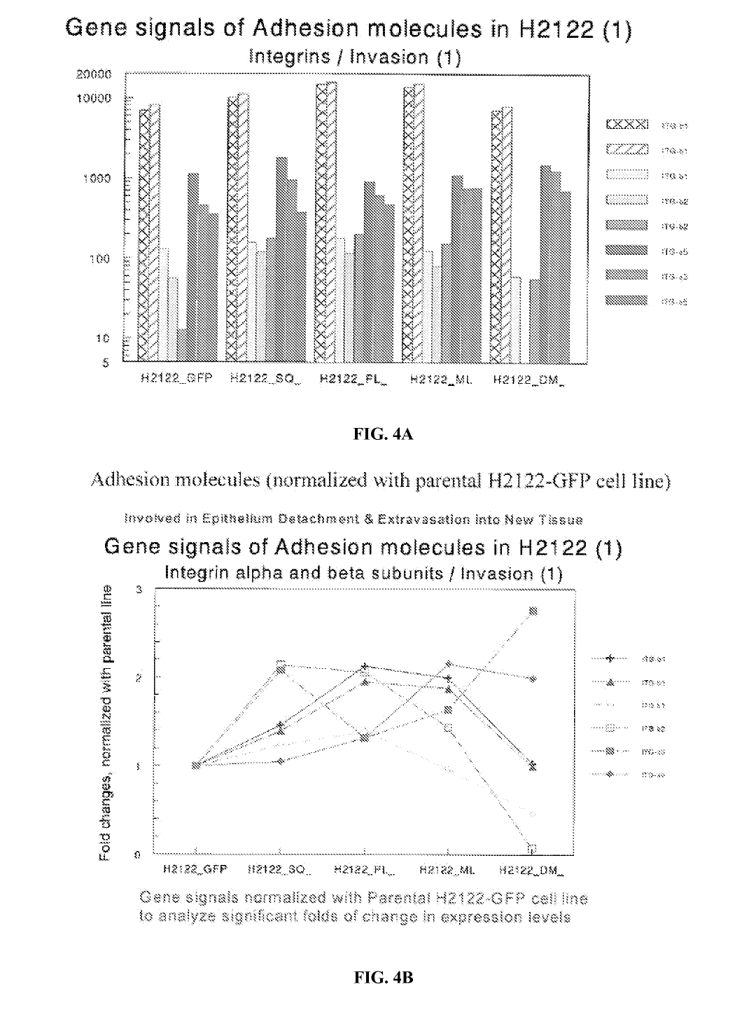

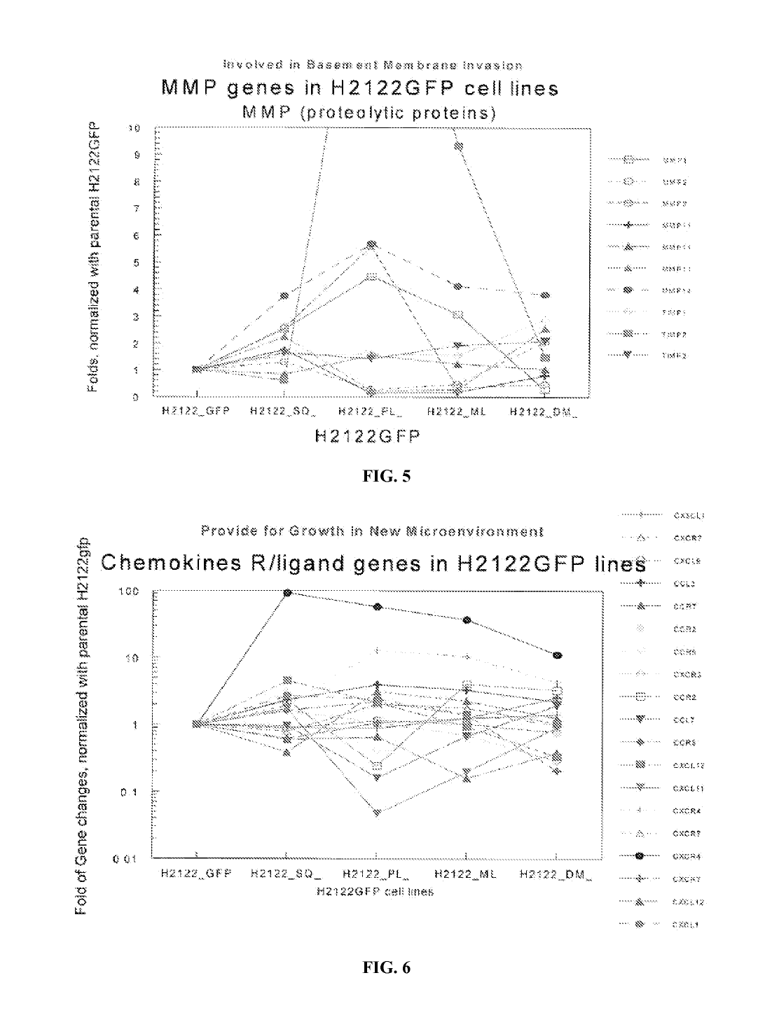

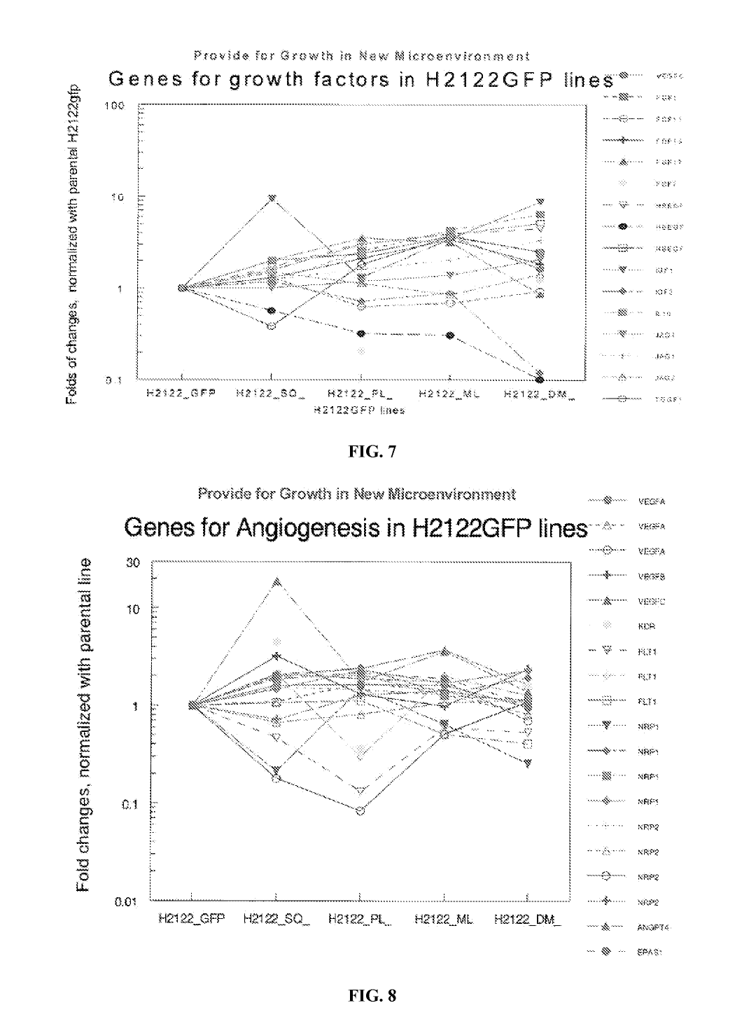

Results: Using the microarray data generated from these parental cells as well as the primary, metastases and distant metastases, many genes have been identified that are normally known to involve in the metastases cascades. The key proteins that are know to be involved in various steps of cancer invasion and metastasis cascades are grouped as follows: (1) Adhesion molecules (FIGS. 4A-B); (2) MMP Proteolytic proteins (FIG. 5); (3) Inflammatory cytokines and chemokines (FIG. 6); (4) Growth factors (FIG. 7); and (5) Angiogenesis (FIG. 8). As shown in FIGS. 4-8, the up- and down-regulations of many key genes previously shown to he involved in tumor invasion and metastatic processes have been confirmed. This has supported and validated the unique animal model, microarray data acquisition and analysis methodology. The results showed that cancer's ability to metastasize is determined by the micro-environment of the new tissues and “cross-talk” between the microenvironment and tumor cells.

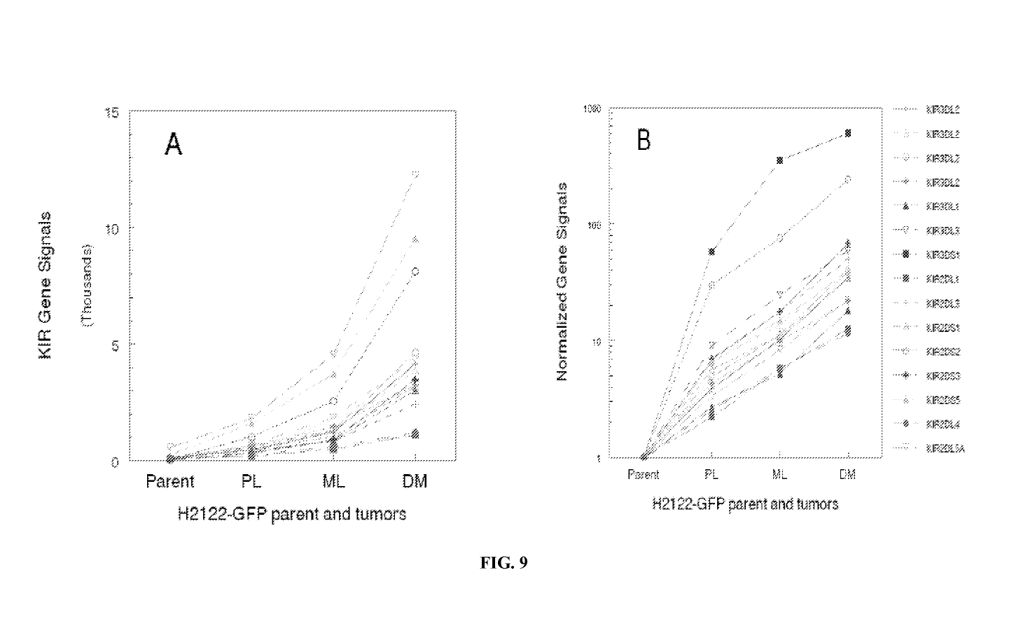

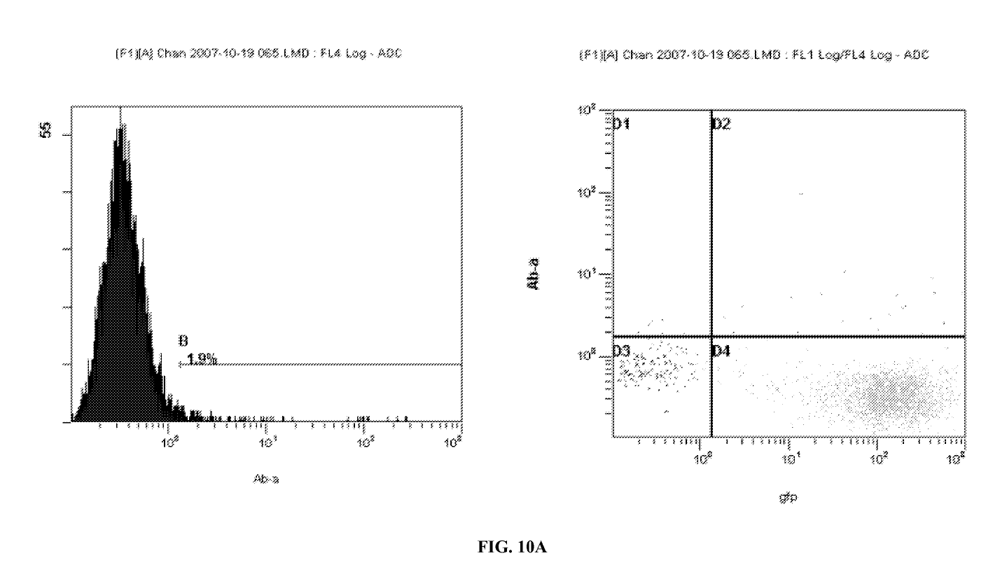

Among the most noticeable differentially regulated genes between primary tumor and their distant metastases are a family of natural killer cell immunoglobulin-like receptor (KIR) genes that are gradually up regulated from the parental, primary, metastasis and distant metastases, as shown in FIGS. 9A-B and Table 2. Levels of KIR gene signals increased significantly and progressively with distance of metastases from primary tumors. In contrast, expression of KIRs was low or undetectable in the parental lung cancer cell line. The possibility that the human tumor samples might be contaminated with rodent NK cells was ruled out by showing that rodent NK cells expressed Killer Cell Lectin-like Receptors (KLR in rat, or Ly49 in mouse) that are functionally equivalent but structurally different from human KIR. The inventors searched for other biomarkers of NK cells in the samples. However, except for KIR genes, other markers commonly found in NK cells such as LIR-1, LAIR, CD94/NKG2A/C/E, SIGLEC, KLRG1, NKR-P1A or CD56 were either barely detectable or failed to change progressively from primary to metastatic cancer cells, as shown in Table 2. The unusual up-regulated expression of human KIR on lung cancer cells after orthotopic implantation in nude rats may be due to the result of tumor cell interaction with their host in the metastatic microenvironment.

TABLE 2
H2122-GFP Ortho Rats

| | | Tissue type ( )* | | | | | | | |
|---|---|---|---|---|---|---|---|---|---|
| | | GFP | PL | ML | DM | GFP | PL | ML | DM |
| Gene Symbol | Systematic | Raw | Raw | Raw | Raw | Normalized | Normalized | Normalized | Normalize [symbol] |
| KIR family | | | | | | | | | |
| KIR2DL1 | 211687_x_at | 88.9 | 197.4 | 509.2 | 1113.7 | 0.1616857 | 0.349672 | 1 | 2.205881 [symbol] |
| KIR2DL2 | 211532_x_at | 47.3 | 274.5 | 196.9 | 951.4 | 0.1194994 | 0.6754465 | 0.5371458 | 2.6176531 |
| KIR2DL3 | 207314_x_at | 48.5 | 343.8 | 858.5 | 2422.5 | 0.0333913 | 0.2305376 | 0.6382254 | 1.816352 |
| KIR2DL4 | 216552_x_at | 73.9 | 221.6 | 283.4 | 659.5 | 0.1921311 | 0.5611344 | 0.7956002 | 1.8672922 |
| KIR2DL4 | 211389_x_at | 104.2 | 261.9 | 606.1 | 1207.4 | 0.1592141 | 0.3897564 | 0.9999999 | 2.009136 |
| KIR2DL5A | 208198_x_at | 545.8 | 1822.8 | 4573.9 | 12231.5 | 0.0865615 | 0.2815622 | 0.783285 | 2.1125906 |
| KIR2DS1 | 216907_x_at | 92.7 | 525.8 | 1018.5 | 3208.9 | 0.062628 | 0.3459821 | 0.7430054 | 2.3609648 |
| KIR2DS2 | 217296_at | 33.6 | 1018.6 | 2548.5 | 8116.3 | 0.01 | 0.2790086 | 0.7739208 | 2.4858358 |
| KIR2DS3 | 207313_x_at | 50.7 | 351.2 | 898 | 3465.3 | 0.0408948 | 0.2759045 | 0.7821291 | 3.044005 |
| KIR2DS4 | 211242_x_at | 10.4 | 299 | 906.8 | 1418.4 | 0.01 | 0.2769255 | 0.9311103 | 1.4688952 |
| KIR2DS5 | 211688_x_at | 247 | 1607.1 | 3731.3 | 9528.8 | 0.0560792 | 0.3553789 | 0.9147593 | 2.3560653 |
| KIR3DL1 | 211410_x_at | 166.4 | 450 | 864.9 | 3033.8 | 0.1130832 | 0.2978525 | 0.6346768 | 2.2453089 |
| KIR3DL2 | 208179_x_at | 94.3 | 633.3 | 1298.2 | 3863.5 | 0.0462162 | 0.3022985 | 0.6870148 | 2.062091 |
| KIR3DL2 | 208426_x_at | 119.6 | 465 | 1225.7 | 4138.2 | 0.059004 | 0.2234329 | 0.6529446 | 2.223341 |
| KIR3DL2 | 217318_x_at | 116.5 | 569 | 1313.9 | 4616.6 | 0.056908 | 0.2707095 | 0.6930291 | 2.4559176 |
| KIR3DL2 | 216676_x_at | 148.6 | 642.6 | 1463.3 | 3252.5 | 0.0723437 | 0.3046956 | 0.7692311 | 1.7244207 |
| KIR3DL3 | 211245_x_at | 74 | 673.2 | 1841.2 | 4371.2 | 0.029375 | 0.2602763 | 0.7892042 | 1.8896945 |
| KIR3DS1 | 208122_x_at | 5.1 | 293.9 | 1791.4 | 3087.3 | 0.01 | 0.1240883 | 0.8385362 | 1.4575067 |
| HLA family | | | | | | | | | |
| HLA-A | | | | | | | | | |
| HLA-A | 203112_s_at | 17021.5 | 16835.6 | 14379.9 | 17659.8 | 1.0327748 | 0.994902 | 0.9421184 | 1.166911 |
| HLA-A | 204599_s_at | 17113 | 18392.8 | 16850.4 | 21016.3 | 0.8642864 | 0.904739 | 0.9189325 | 1.155931 |
| HLA-B | | | | | | | | | |
| HLA-B | 217456_x_at | 8665.3 | 7723.8 | 7590.7 | 9984.4 | 0.8444651 | 0.7331166 | 0.79877 | 1.0596547 |
| HLA-B | 224756_s_at | 1729.4 | 1466.2 | 1386.2 | 2102.7 | 0.7711151 | 0.6367381 | 0.667408 | 1.021045 [symbol] |
| HLA-C | | | | | | | | | |
| HLA-C | 211146_at | 14507.5 | 12341.1 | 13923.5 | 17430.4 | 0.8749222 | 0.7248942 | 0.9067072 | 1.144796 [symbol] |
| HLA-C | 214669_x_at | 7156.1 | 6005.9 | 6154.3 | 8988.1 | 0.848537 | 0.6936116 | 0.7879793 | 1.160663 [symbol] |
| HLA-C | 214768_x_at | 1548.6 | 1244.1 | 1250.5 | 1491.3 | 0.9804071 | 0.7671253 | 0.8548554 | 1.028197 [symbol] |
| HLA-C | 217436_x_at | 8725.5 | 7284.8 | 7101.7 | 10761.6 | 0.9210013 | 0.7489131 | 0.8094202 | 1.2370607 |
| HLA-D | | | | | | | | | |
| HLA-DPB2 | 1553103_at | 24.4 | 10.3 | 41.9 | 79 | 1 | 0.4111415 | 1.8542404 | 3.5259936 |
| HLA-DRB1; | 1553348_a_at | 1.2 | 2.6 | 1.9 | 10.2 | 0.1118868 | 0.2361103 | 0.1912903 | 1.0357192 |
| HLA-DRB3 | | | | | | | | | |
| HLA-DQA1 | 1567627_at | 15.2 | 5.5 | 1.2 | 38.5 | 1.4172329 | 0.499464 | 0.120815 | 3.9093323 |
| HLA-DOA | 1567628_at | 5.5 | 5.5 | 16.8 | 21.2 | 0.2571819 | 0.2504865 | 0.8482596 | 1.0795876 |
| HLA-DRB1 | 201137_s_at | 4.8 | 1 | 24.8 | 39.5 | 0.322615 | 0.0654617 | 1.7998515 | 2.8912427 |
| HLA-DMA | 202585_s_at | 418.1 | 453.6 | 400.1 | 628.7 | 1.0081686 | 1.0652951 | 1.0417496 | 1.650976 |
| HLA-DRB6 | 203290_at | 18.7 | 4.4 | 47.6 | 39.3 | 0.8203465 | 0.1879976 | 2.2547815 | 1.8775543 |
| HLA-DRB6 | 203932_at | 69.8 | 3.5 | 24.6 | 34.1 | 6.5080824 | 0.3178408 | 2.4767065 | 3.4625514 |
| HLA-DOA | 204670_x_at | 62.9 | 97.5 | 57.4 | 13.8 | 0.6146694 | 0.9279817 | 0.605682 | 0.1468636 |
| HLA-DOA | 205671_s_at | 27.8 | 19.4 | 15.6 | 36.3 | 1.0522944 | 0.7152174 | 0.6376155 | 1.4963849 |
| HLA-DPA2 | 206313_at | 58.6 | 47.7 | 50.9 | 66 | 1.0729606 | 0.8506452 | 1.0063424 | 1.3160559 |
| HLA-DRB4 | 208306_x_at | 46.5 | 7.1 | 1.6 | 13.4 | 2.001677 | 0.2976756 | 0.0743709 | 0.6281887 |
| HLA-DRB4 | 208894_at | 1.1 | 1.6 | 2.3 | 2.2 | 0.1025629 | 0.1452986 | 0.231562 | 0.223390 [symbol] |
| HLA-DQB2 | 209312_x_at | 56.7 | 53.4 | 49.6 | 58.1 | 1.3349057 | 1.2244828 | 1.2609301 | 1.489662 [symbol] |
| HLA-DRB1 | 209480_at | 73.3 | 34.7 | 39.1 | 34 | 1.736143 | 0.8004881 | 1 | 0.877010 [symbol] |
| HLA-DQA1 | 209728_at | 23.3 | 10 | 36.5 | 17.8 | 1.4706054 | 0.6147296 | 2.4875677 | 1.223501 [symbol] |
| HLA-DPA1 | 209823_x_at | 18.5 | 4.9 | 4.5 | 16.5 | 1.7249216 | 0.4449771 | 0.4530561 | 1.6754282 |
| HLA-DQB1 | 210268_at | 5.1 | 6 | 2.2 | 20.9 | 0.4755189 | 0.5448698 | 0.2214941 | 2.122208 [symbol] |
| HLA-DQB1 | 210747_at | 3.1 | 2.2 | 2 | 2.7 | 0.2890409 | 0.1997856 | 0.2013583 | 0.27416 [symbol] |
| HLA-DQA1; | 210982_s_at | 2.4 | 1.9 | 5.1 | 1.6 | 0.2237736 | 0.1725421 | 0.5134635 | 0.162465 [symbol] |
| HLA-DQA2 | | | | | | | | | |
| HLA-DPA1 | 211142_x_at | 26.2 | 15.2 | 37.9 | 21.1 | 0.8907843 | 0.5033368 | 1.3914008 | 0.781264 [symbol] |
| HLA-DPA1 | 211654_x_at | 25.1 | 31.6 | 49.3 | 39.4 | 0.8490257 | 1.0410656 | 1.800677 | 1.4514016 |
| HLA-DQB1 | 211656_x_at | 28 | 4.3 | 6.5 | 82.6 | 0.7692978 | 0.1150665 | 0.1928376 | 2.4715009 |
| HLA-DQB1 | 211990_at | 4.6 | 74.4 | 86.6 | 63.7 | 0.0770719 | 1.2141018 | 1.5667439 | 1.16231 |
| HLA-DOA | 211991_s_at | 9.3 | 13.3 | 4.9 | 8.7 | 0.7711257 | 1.0740829 | 0.4387126 | 0.7856077 |
| HLA-DRA | 212671_s_at | 8.3 | 5.4 | 4.6 | 4 | 0.7738837 | 0.4903828 | 0.463124 | 0.4061644 |
| HLA-DQB1 | 212998_x_at | 16.6 | 2.6 | 10.4 | 2.5 | 1.5477674 | 0.2361103 | 1.0470628 | 0.2538528 |
| HLA-DQB1 | 213537_at | 10 | 4.9 | 4.2 | 6.9 | 0.93239 | 0.4449771 | 0.4228523 | 0.7006337 |
| HLA-DRB4 | 213831_at | 2.5 | 2.7 | 3.2 | 3.5 | 0.2330975 | 0.2451914 | 0.3221732 | 0.3553939 |
| HLA-DQB1 | 215193_x_at | 2.9 | 3.4 | 2.3 | 2 | 0.2703931 | 0.3087596 | 0.231562 | 0.2030822 |
| HLA-DRB1 | 215536_at | 113.1 | 85 | 80.3 | 52.7 | 1.4513392 | 1.0623536 | 1.1126631 | 0.7364804 |
| HLA-DRA | 215666_at | 2.6 | 7.7 | 40.8 | 36.1 | 0.2424214 | 0.6992496 | 4.107708 | 3.6656332 |
| HLA-DRB4 | 215669_at | 65.8 | 88.7 | 47.3 | 77.3 | 0.801275 | 1.052018 | 0.6219545 | 1.0251312 |
| HLA-DOA | 216631_s_at | 43.6 | 7.4 | 12.4 | 13.2 | 1.7138364 | 0.2833078 | 0.5263158 | 0.5650685 |
| HLA-DOB | 216946_at | 102.2 | 44 | 45.6 | 5.8 | 3.3273997 | 1.3952454 | 1.6031003 | 0.205648 [symbol] |
| HLA-DRB5 | 217001_x_at | 61.9 | 116.8 | 49.3 | 91.7 | 0.9258338 | 1.7014889 | 0.7962164 | 1.493674 [symbol] |
| HLA-DMB | 217323_at | 168.6 | 110.5 | 100.9 | 197.9 | 1.4589901 | 0.9313243 | 0.9428178 | 1.865025 [symbol] |
| HLA-DQA1 | 217362_x_at | 6.6 | 11.7 | 6.3 | 3.3 | 0.6153774 | 1.0624962 | 0.6342785 | 0.335085 [symbol] |
| HLA-DPB1 | 221491_x_at | 44.8 | 35.4 | 6.9 | 10.5 | 3.3619213 | 2.5873587 | 0.559114 | 0.858110 [symbol] |
| HLA-E | | | | | | | | | |
| HLA-E | 200904_at | 1113.5 | 1031.2 | 1197 | 1293 | 0.8046461 | 0.7257741 | 0.9340081 | 1.01755 [symbol] |
| HLA-E | 203307_at | 3762.3 | 3389.8 | 3788.3 | 3614.2 | 0.9558659 | 0.838806 | 1.0392733 | |
| HLA-E | 217456_x_at | 523.1 | 448.1 | 662.2 | 1103.7 | 0.6900773 | 0.5757472 | 0.9432873 | 1.585653 [symbol] |
| HLA-F | | | | | | | | | |
| HLA-F | 203146_s_at | 39 | 49.6 | 20.2 | 40.9 | 0.8073076 | 1 | 0.4515103 | 0.922023 [symbol] |
| HLA-F | 204806_x_at | 2689.1 | 2822.7 | 2424.1 | 2354.9 | 0.9999999 | 1.0223547 | 0.9733865 | 0.9536954 |
| HLA-F | 221875_x_at | 2148.7 | 2797.3 | 2033.1 | 3112.6 | 0.6338817 | 0.8037396 | 0.6476389 | 1 |
| HLA-G | | | | | | | | | |
| HLA-G | 211528_x_at | 1520.4 | 1439 | 1477.9 | 1367.2 | 0.9527327 | 0.8782494 | 1 | 0.9330168 |
| HLA-G | 211529_x_at | 2447.6 | 2506.4 | 2425.1 | 2988.1 | 0.7831908 | 0.7811264 | 0.8379123 | 1.0412775 |
| HLA-G | 211530_x_at | 3045.3 | 2828.4 | 2619.2 | 2627.6 | 1.0302072 | 0.9319211 | 0.9567642 | 0.9680504 |
| HLA-G | 217436_x_at | 1068 | 1575.4 | 973.6 | 1074.8 | 0.7479075 | 1.0745122 | 0.7362054 | 0.819688 |
| HLA-J | | | | | | | | | |
| HLA-J | 208812_x_at | 844.7 | 999.5 | 853.6 | 1216.1 | 0.6392197 | 0.7366723 | 0.6974994 | 1.0022156 |
| NK cell marker | | | | | | | | | |
| except KIR | | | | | | | | | |
| CD2 | 205831_at | 110.6 | 81 | 4.2 | 31.9 | 1.7303565 | 1.2342677 | 0.0709531 | 0.5435198 |
| CD244 | 234320_at | 3.4 | 3.7 | 4.2 | 15.1 | 0.3170126 | 0.3360031 | 0.4228523 | 1.5332706 |
| CD244 | 220307_at | 14 | 29 | 20.2 | 22.4 | 0.9028132 | 1.821427 | 1.4065754 | 1.5731211 |
| CD5 | 230489_at | 7.7 | 67.3 | 12.6 | 9.4 | 0.4054088 | 3.4511309 | 0.7163327 | 0.5389823 |
| CD5 | 206485_at | 149.6 | 124.2 | 58.9 | 34.6 | 1.3825376 | 1.1179202 | 0.5877633 | 0.3482296 |
| CD56 | 217359_s_at | 24 | 5.8 | 6.4 | 5.5 | 1.2501318 | 0.29425 | 0.3599701 | 0.3119978 [symbol] |
| CD56 | 209968_s_at | 10.8 | 9.9 | 7.3 | 44 | 1.0069813 | 0.8990352 | 0.7349576 | 4.467808 [symbol] |
| CD57 | 219521_at | 29.3 | 7.1 | 14.6 | 7.6 | 2.7319026 | 0.6447626 | 1.4699152 | 0.771712 [symbol] |
| FCGR3B | 204007_at | 8.2 | 35.8 | 33.3 | 45.9 | 0.1925843 | 0.818906 | 0.8444873 | 1.17398 [symbol] |
| FCGR3B | 204006_s_at | 4.7 | 2.4 | 4.5 | 30 | 0.4382233 | 0.2179479 | 0.4530561 | 3.046233 [symbol] |
| GEM | 204472_at | 470.3 | 563.3 | 574.6 | 767 | 0.8674082 | 1.0118873 | 1.1443422 | 1.540593 [symbol] |
| IL2 | 217181_at | 3 | 5.3 | 2.9 | 3.3 | 0.279717 | 0.4813017 | 0.2919695 | 0.3350856 [symbol] |
| IL2 | 207849_at | 1.2 | 1.2 | 11.8 | 1.5 | 0.1118868 | 0.108974 | 1.1880137 | 0.1523116 |
| ITGAL | 1554240_a_at | 5.8 | 33.1 | 5.5 | 14.8 | 0.3598505 | 2.0001655 | 0.3684669 | |
| ITGAM | 205786_s_at | 64.4 | 42.8 | 11.9 | 43.5 | 2.0825372 | 1.3480144 | 0.4155235 | 1.531935 [symbol] |
| ITGAM | 205785_at | 34.3 | 82.3 | 80.7 | 44.3 | 0.5033543 | 1.1763145 | 1.2787775 | 0.707990 [symbol] |
| ITGAX | 210184_at | 7 | 19.6 | 10.5 | 9.7 | 0.6136249 | 1.67342 | 0.9938849 | 0.926021 [symbol] |
| ITGAX | 1563003_at | 52.9 | 3.6 | 7.3 | 2.4 | 4.2403502 | 0.2810557 | 0.6318453 | 0.209508 [symbol] |
| ITGB2 | 202803_s_at | 12.6 | 199.2 | 154.9 | 55.7 | 0.1204298 | 1.8543713 | 1.5986621 | 0.5797796 |
| ITGB2 | 1555349_a_at | 56.4 | 116.1 | 81.1 | 4.3 | 1.0881757 | 2.1817048 | 1.6895946 | 0.0903509 |
| KLRD1 | 210606_x_at | 12.2 | 31.2 | 33.6 | 4.3 | 0.651973 | 1.6239334 | 1.9388794 | 0.2502548 |
| KLRD1 | 207796_x_at | 19.2 | 7.6 | 6.9 | 10.3 | 1.790189 | 0.6901684 | 0.6946859 | 1.0458733 |
| KLRD1 | 207795_s_at | 63.2 | 10.9 | 21.8 | 8.2 | 5.892705 | 0.9898468 | 2.1948047 | 0.832637 |
| KLRG1 | 210288_at | 176.2 | 142.6 | 123.9 | 92.9 | 1.7640471 | 1.3904895 | 1.339422 | 1.0128946 |
| KLRK1 | 205821_at | 50.2 | 39.1 | 9.5 | 8.8 | 4.6805983 | 3.5507348 | 0.9564517 | 0.8935617 |
| LTBR | 203005_at | 1866.8 | 2512.2 | 2500.1 | 3314.5 | 0.731796 | 0.9591583 | 1.0582577 | 1.4149936 |
| NCR1 | 217095_x_at | 18.8 | 17.2 | 3.5 | 6.9 | 1.1222394 | 1 | 0.2255992 | 0.4485605 |
| NCR1 | 217088_s_at | 5.5 | 4.3 | 2.8 | 6.5 | 0.5128145 | 0.3904901 | 0.2819015 | 0.6600171 |
| NCR1 | 207860_at | 4.2 | 20.5 | 18.5 | 3.2 | 0.3916038 | 1.8616385 | 1.8625637 | 0.3249316 |
| NCR2 | 221075_s_at | 26.8 | 9.3 | 11.8 | 11.2 | 2.4988053 | 0.8445482 | 1.1880137 | 1.1372603 |
| NCR2 | 221074_at | 9.2 | 3.1 | 9.1 | 4.1 | 0.8577988 | 0.2815161 | 0.9161801 | 0.4163185 |
| NCR3 | 211583_x_at | 7.4 | 16.1 | 10.8 | 4.2 | 0.6899687 | 1.4620674 | 1.0873345 | 0.4264726 |
| NCR3 | 211010_s_at | 25 | 44.7 | 14.2 | 40.5 | 1.3010777 | 2.2657638 | 0.7979825 | 2.2954216 |
| NCR3 | 210763_x_at | 8 | 4.2 | 8.5 | 3.9 | 0.745912 | 0.3814089 | 0.8557726 | 0.3960103 |
| PTPRC | 212588_at | 1.3 | 23.5 | 31.8 | 18.3 | 0.0669672 | 1.1790462 | 1.7688375 | 1.026631 [symbol] |
| PTPRC | 207238_s_at | 5.1 | 6.6 | 3.1 | 8 | 0.4755189 | 0.5993568 | 0.3121053 | 0.812328 [symbol] |
| PTPRC | 1552480_s_at | 14.2 | 18.3 | 2.1 | 7.1 | 1.3239938 | 1.6618528 | 0.2114261 | 0.720941 [symbol] |
| ITIM related genes | | | | | | | | | |
| LILRB1 | 206856_at | 12.9 | 1.2 | 4.7 | 4 | 1.2027831 | 0.108974 | 0.4731919 | 0.406164 [symbol] |
| LILRA1 | 211135_x_at | 39.3 | 63.9 | 23.8 | 27 | 1.3451742 | 2.1302507 | 0.8796395 | 1.0064541 [symbol] |
| LILRB1 | 207697_x_at | 10.1 | 14.3 | 22.9 | 5.1 | 0.560719 | 0.7732213 | 1.3727808 | 0.308346 |
| LILRB1 | 213975_s_at | 4.7 | 11.1 | 11.8 | 4.5 | 0.4382233 | 1.0080092 | 1.1880137 | 0.456934 [symbol] |
| LILRB2 | 211133_x_at | 3.3 | 25.8 | 24.2 | 1.9 | 0.3076887 | 2.34294 | 2.4364347 | 0.192928 [symbol] |
| LILRB2 | 211336_x_at | 3.8 | 3.5 | 4.6 | 8.3 | 0.3543082 | 0.3178408 | 0.463124 | 0.842791 [symbol] |
| LILRB3 | 207872_s_at | 14.7 | 18.9 | 7.6 | 43.1 | 0.9569163 | 1.198291 | 0.53421 | 3.055470 [symbol] |
| LILRB3 | 210146_x_at | 12.5 | 86.4 | 83.7 | 37.2 | 0.262353 | 1.7661744 | 1.8968947 | 0.850282 [symbol] |
| LILRB3 | 210152_at | 22.9 | 36.9 | 10.8 | 26.7 | 1 | 1.5694041 | 0.5092489 | 1.2697554 |
| LILRB3 | 210225_x_at | 12.7 | 11.2 | 36 | 15.4 | 0.7697802 | 0.6611879 | 2.3561738 | 1.016548 |
| LILRB4 | 210784_x_at | 30.7 | 56.2 | 72.9 | 42.2 | 0.6910178 | 1.2320577 | 1.7718225 | 1.0344453 |
| LILRB5 | 229937_x_at | 6.6 | 4.4 | 27.1 | 11.1 | 0.4949835 | 0.3213982 | 2.194613 | 0.9065965 |
| LYZ; LILRB1 | 207104_x_at | 2.4 | 40.2 | 3.1 | 5.1 | 0.2237736 | 3.650628 | 0.3121053 | 0.5178596 |
| Killer cell lectin-like | | | | | | | | | |
| receptors | | | | | | | | | |
| KLRA1 | 214470_at | 49.7 | 10.1 | 38.8 | 28.8 | 2.2706184 | 0.4494207 | 1.9140853 | 1.4329284 |
| KLRB1 | 205821_at | 3.4 | 25.3 | 29 | 1.5 | 0.1833316 | 1.328687 | 1.6884885 | 0.0880834 |
| KLRB1 | 207229_at | 2.7 | 7.4 | 10.8 | 2.3 | 0.2517453 | 0.6720061 | 1.0873345 | 0.2335445 |
| KLRC2 | 220646_s_at | 11 | 1.6 | 2 | 14.4 | 1.025629 | 0.1452986 | 0.2013583 | 1.4621918 |
| KLRC3 | 210690_at | 41.8 | 47.1 | 24.2 | 14.4 | 3.2548668 | 3.5720842 | 2.0347643 | 1.2211349 |
| KLRC4 | 207723_s_at | 0.5 | 25.8 | 26.3 | 1.4 | 0.0466195 | 2.34294 | 2.6478608 | 0.1421575 |
| KLRC4 | 242873_at | 35.6 | 10.1 | 40.9 | 15.9 | 1.0180424 | 0.2813074 | 1.2629352 | 0.4951734 |
| KLRD1 | 207795_s_at | 12.2 | 31.2 | 33.6 | 4.3 | 0.651973 | 1.6239334 | 1.9388794 | 0.2502548 |
| KLRD1 | 210288_at | 19.2 | 7.6 | 6.9 | 10.3 | 1.790189 | 0.6901684 | 0.6946859 | 1.0458733 |
| KLRD1 | 210606_x_at | 63.2 | 10.9 | 21.8 | 8.2 | 5.892705 | 0.9898468 | 2.1948047 | 0.832637 [symbol] |
| KLRF1 | 206785_s_at | 63.6 | 261.8 | 145.5 | 89 | 0.4219355 | 1.6916187 | 1.0423025 | 0.643018 [symbol] |
| KLRG1 | 207796_x_at | 176.2 | 142.6 | 123.9 | 92.9 | 1.7640471 | 1.3904895 | 1.339422 | 1.012894 [symbol] |
| KLRK1 | 1555691_a_at | 43 | 2.9 | 39.2 | 6.6 | 1.523827 | 0.1000942 | 1.500013 | 0.254715 [symbol] |
| KLRK1 | 242628_at | 50.2 | 39.1 | 9.5 | 8.8 | 4.6805983 | 3.5507348 | 0.9564517 | 0.893561 [symbol] |
| Granzymes | | | | | | | | | |
| GZMA | 210321_at | 29.2 | 33.4 | 30.9 | 2.7 | 1.6984421 | 1.8921618 | 1.9407436 | 0.1710314 |
| GZMB | 206666_at | 29.1 | 5.2 | 10 | 40.1 | 2.713255 | 0.4722205 | 1.0067912 | 4.07179 [symbol] |
| GZMH | 205488_at | 10 | 4.8 | 9.6 | 19.2 | 0.93239 | 0.4358959 | 0.9665197 | 1.949589 [symbol] |
| GZMK | 210164_at | 28.4 | 20.5 | 11.3 | 14.7 | 1.7740129 | 1.2472003 | 0.762182 | |
| GZMM | 207460_at | 4.3 | 8.7 | 5.7 | 9.8 | 0.4009277 | 0.7900612 | 0.573871 | 0.995102 [symbol] |
| Perforins | | | | | | | | | |
| PRF1 | 1553681_a_at | 50.1 | 95.7 | 83.6 | 87.3 | 0.732999 | 1.3637081 | 1.320729 | 1.3909904 |
| PRF1 | 214617_at | 52.9 | 111 | 97.5 | 86.6 | 0.5805462 | 1.186446 | 1.155387 | 1.0350069 |

[symbol]

indicates data missing or illegible when filed

When normalized with parental H2122-GFP as shown in FIG. 4-8, a broad spectrum of genes that are known to involve in the invasion and metastatic process such as proliferative growth factors/receptors, MMPs, inflammatory cytokines/chemokines, apoptosis and angiogenesis, are found to be up-regulated or down-regulated progressively from parental→primary→metastases→distant metastases, while subcutaneous tumors responded differently (FIGS. 4-8). The overall changes in KIR gene families in the H2122-GFP cell lines was much higher than those key proteins mentioned above (FIG. 9B), suggesting that the significantly increased expressions of the KIR proteins plays important in the tumor metastatic process.

### Example 3

### Detection of KIR Proteins on H2122 Cells

To further confirm the elevated KIR protein expression, GFP tagged H2122 parental and metastatic cells were stained with commercial anti-KIR antibodies conjugated with APC fluorescent dyes (Miltenyi Biotec) and analyzed by flow cytometry. Four different KIR subtypes were used for the staining studies, which may reflect different KIR expressions among these H2122-GFP cell lines. Antibodies directed against KIR subtypes KIR2DL1, KIR2DL2, KIR3DL1 and KIR2DS4 were tested and found to be positive in the samples. The double positive cells (KIR+ and GFP+) were enriched using flow cytometry cell sorter. The KIR-enriched populations were re-stained with anti-KIR antibodies.

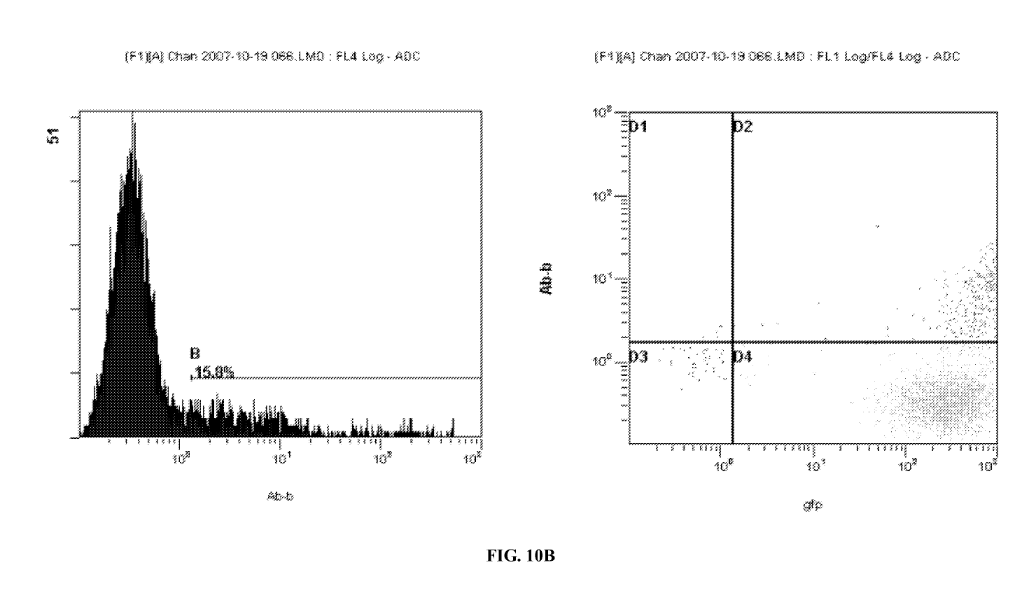

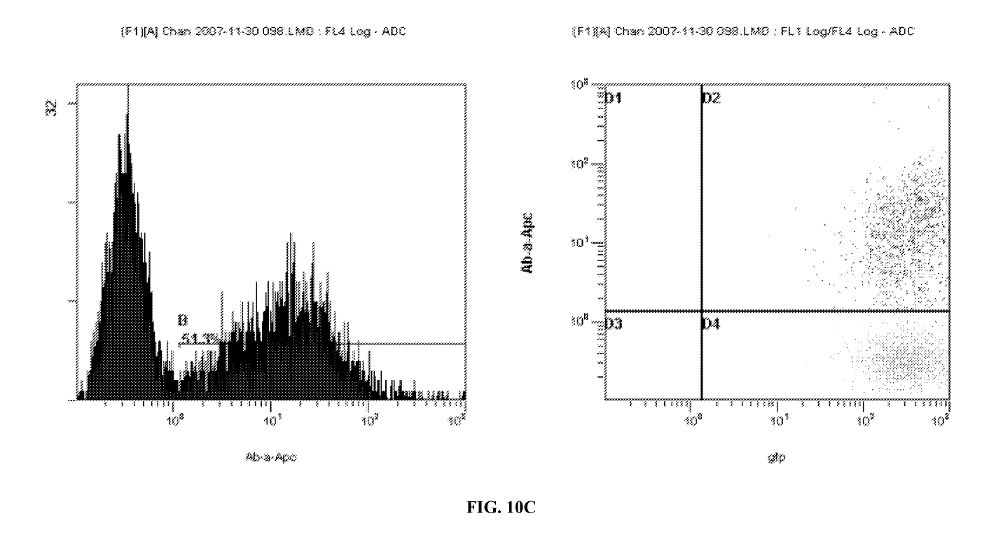

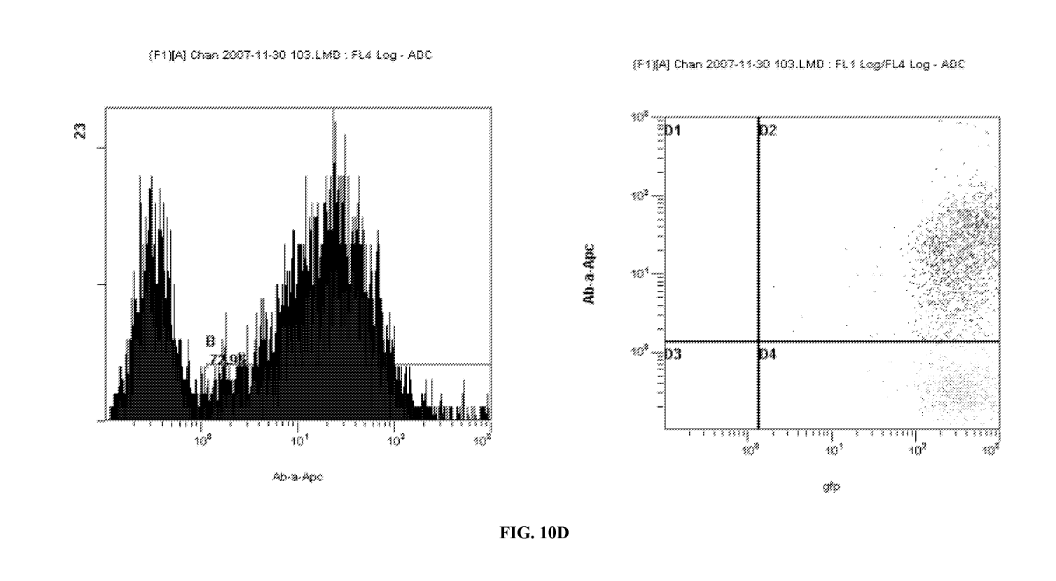

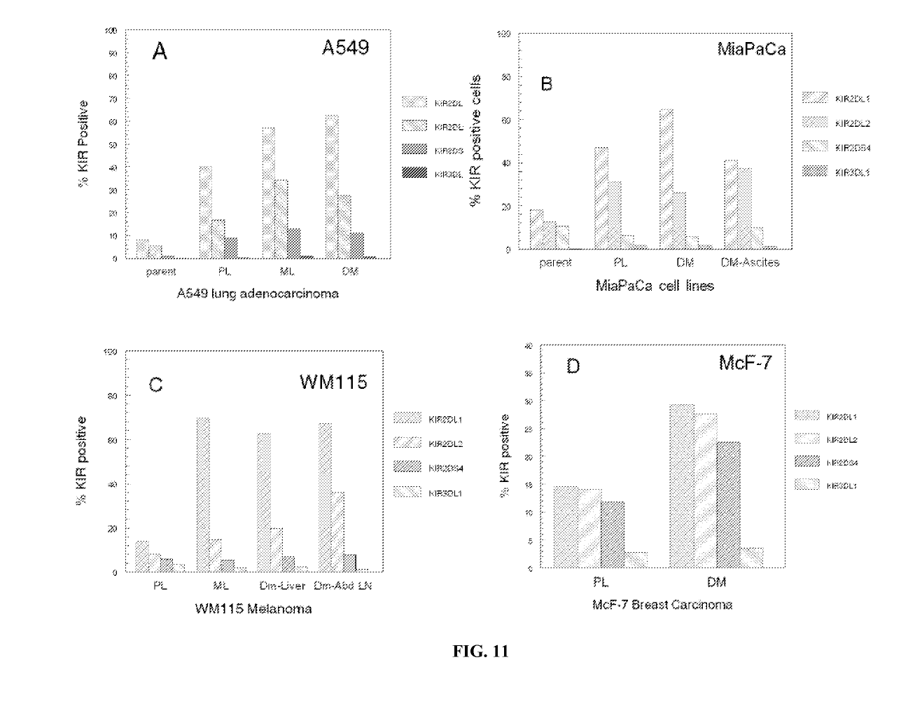

Among the four subtypes, anti-KIR2DL1 provided the strongest mean fluorescent intensity and reacted with a higher percentage of cells in all of the samples tested. The results are shown in FIGS. 10A-D. Only 2% positive cells were detected in parental H2122 cells, 15% in cells derived from the primary tumor, 30% in cells derived from contra-lateral lung metastases and more than 60% in cells derived from distant metastases. The numbers for double positive cells (in pink color) (GFP and KIR positive as indicated by APC) and mean fluorescent intensity increased significantly from parental→primary→metastases→distant metastases in a manner similar to the microarray data described above. In summary, human KIR gene family expression is detectable at significant levels only in cancer cells derived from orthotopic implantations and KIR levels unexpectedly increase progressively with increase in metastatic aggressiveness.

### Example 4

### KIR Protein Expression on Other Cancers After Orthotopic Implantation

KIR protein expression after orthotopic implantation into athymic rodents was evaluated using several other human cancer cell lines. Data from another NSCLC adenocarcinoma (A549) grown in lungs of rats, and the MiaPaCa pancreatic tumor grown in the pancreas of mice are shown in FIGS. 11A and B. Significantly elevated KIR protein levels were detected in the tumor cells compared to parental cells that had not been passaged in rodents. Since the lung is a common site for metastases of many cancers, human melanoma WM115 cells (FIG. 11C) and human breast cancer MCF-7 cells (FIG. 11D) were implanted intratracheally in the left lung of athymic nude rats. Breast cancer cells metastasized to the thymus, and melanoma cells metastasized to the contralateral lung, liver and abdominal lymph nodes. KIR expression was again significantly elevated in the metastases compared to the primary site. These studies demonstrate that KIR over-expression reflects the degree of aggressive and metastatic behavior of a variety of cancers.

The inventors have further demonstrated metastasis of KIR positive H2122-PL-GFP implanted orthotopiclly in a nude mouse (FIG. 2). This KIR positive cancer cell spreads nearly everywhere, including bone marrow, where NK cells are produced. H2122-PL-GFP obtained from the bone marrow are still alive, which indicates that these KIR-positive H2122-PL-GFP are very resistant to NK cells. This demonstrates that KIR-expressed cancer cells have acquired the mechanism of immuno-escape and are capable of metastasizing to distant sites.

### Example 5

### Resistance to Cytolytic Killing by NK Cells

Metastatic cancer cells over-expressed with KIR are demonstrated as more resistant to cytolytic killing by human NK cells than the parental cell lines. To demonstrate that KLR-over-expressed human lung cancer cells or melanoma cells are more resistant to rodent NK cells, KIR protein expression after passage as tumors in athymic nude rats was evaluated in another orthotopic NSCLC cell line (A549 adenocarcinoma). KIR expression was also evaluated in a pancreatic carcinoma MiaPaCa in an orthotopic pancreatic cancer model in athymice nude mice. NK cells are isolated from human peripheral blood using magnetic microbeads tagged with antibodies against CD56 and CD16 (both are NK cells markers) and use the non-radioactive Cytotoxicity Assay (CytoTox 96 from Promega). The NK cells are used to analyze killing, first of the KIR-over-expressing human lung cancer cells that have been selected, and second of KIR-overexpressing melanoma cells. K562 human erythroleukemia cells are used as a positive control. It is then examined whether killing of lung cancer cells varies with KIR expression. Antibodies against KIR subtypes are used to delineate the diversity and expression level of KIR among metastatic cells. Cytolytic killing is similarly analyzed using rodent NK cells.

### Example 6

### In Vitro Anti-Proliferation by NK Cells

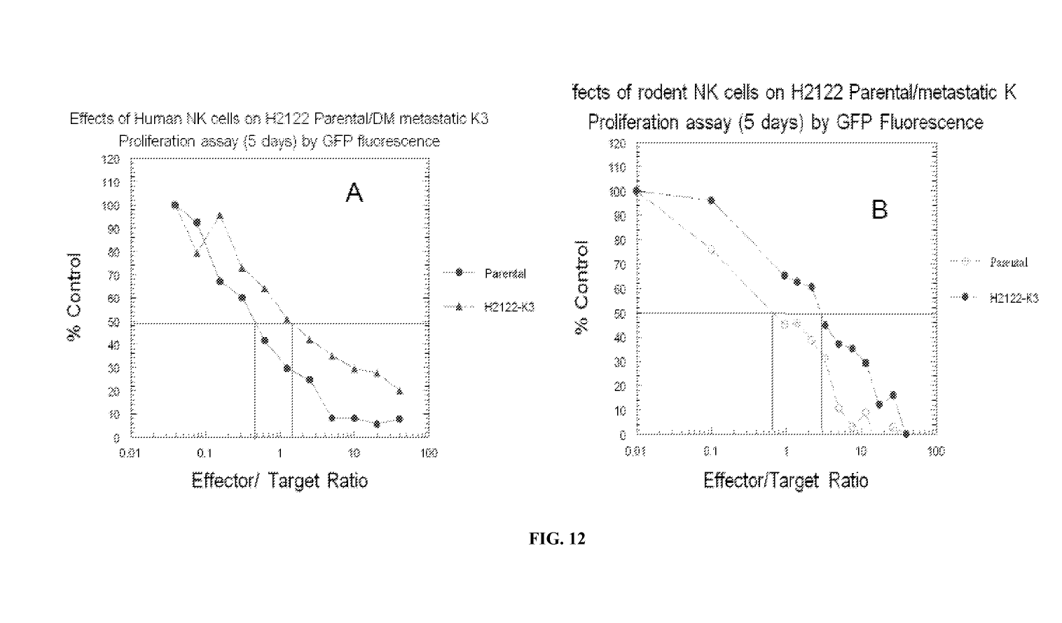

To test whether expression of KIR on the cancer cell surface confers immuno-resistance that leads to increasing metastatic behavior, H2122-GFP parental cells (P) and a metastatic subclone (K3) with high KIR expression were exposed to various amounts of freshly isolated NK cells. NK cells kill cancer cells by apoptosis and thereby inhibit tumor cell proliferation depending on the effector/target ratio (ETR). As shown in FIG. 12A, when assayed on day 5, human NK cells inhibited H2122 parental cells with IC50=0.45 ETR and metastatic subclones K3 with IC50=1.6 ETR. K3 is relatively 3.6 fold more resistant than its parental cells to human NK cell killing. Similarly, as shown in FIG. 12B, on day 5 rodent NK cells inhibited H2122 parental cells with IC50=0.65 ETR, and metastatic K3 subclones with IC50=3 ETR. Again, K3 is relatively 4.6 fold more resistant than its parental cells to rodent NK cell killing. These in vitro cell-killing assays show that metastatic cells with higher KIR expression are slightly more resistant to killing by human or rodent NK cells than their parental cells.

### Example 7

### In Vitro Selection of Immuno-Resistant Lung Cancer Cells with Elevated KIR Expression

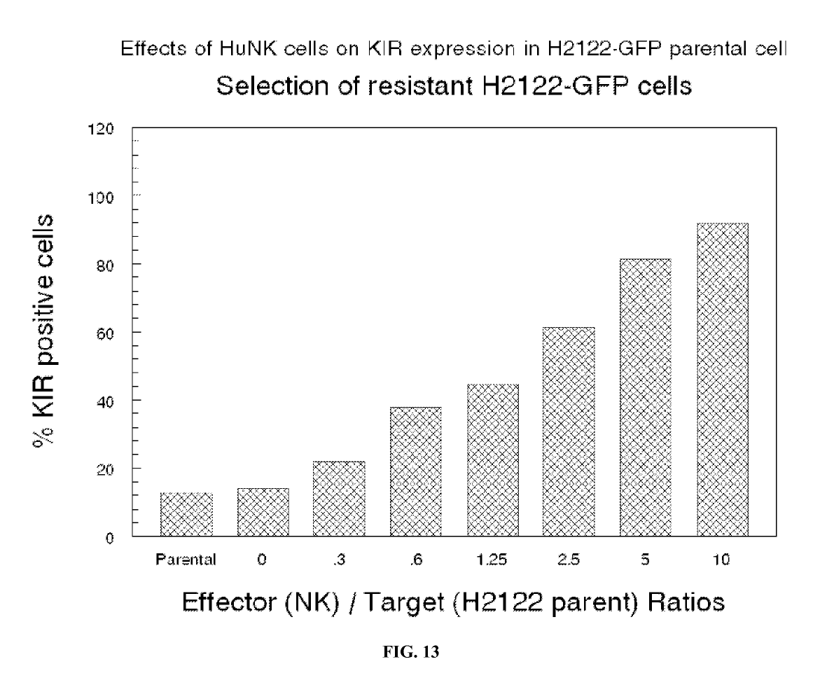

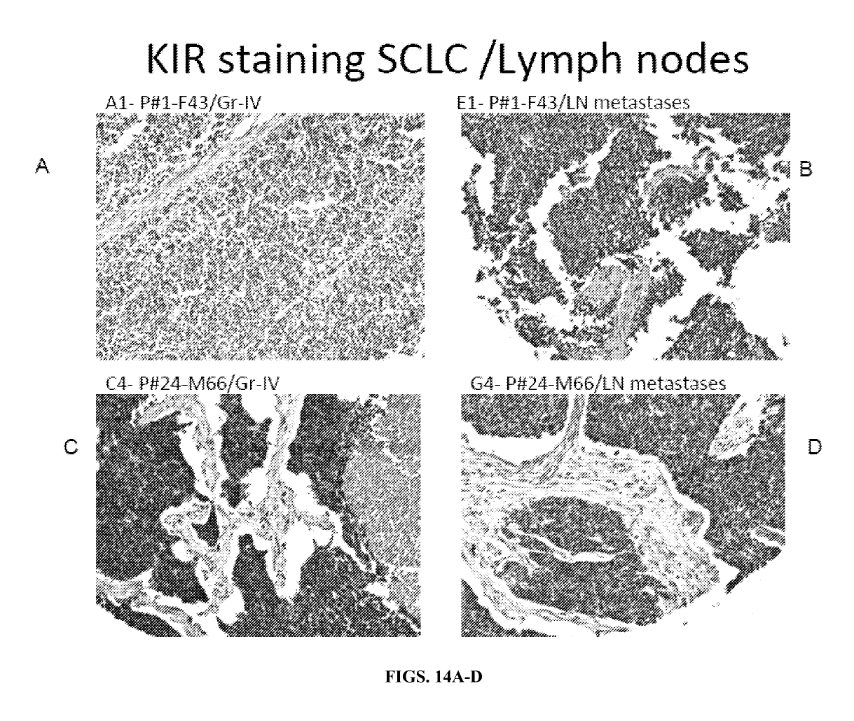

Continuous and repeated exposure of many cancer cells to chemotherapeutic agents in cell culture leads to establishment of multi-drug resistant cell lines with elevated expression of P-glycoproteins (Gottesman and Pastan, 1993; Mealey et al., 1998; Cole et al., 1992). Similarly, co-culturing of a murine melanoma cell line B16 with syngeneic lymphocytes resulted in the selection of a resistant B16-F10 cell line that was found to be very aggressive and highly metastatic (Gottesman and Pastan, 1993; Mealey et al., 1998; Cole et al., 1992). H2122-GFP parental cells were therefore continuously co-cultured with various amounts of human NK cells to isolate subclones that are resistant to NK killings. These surviving NK-resistant parental cells were analyzed for KIR expression by immuno-fluorescent flow cytometry with anti-KIR antibodies. As shown in FIG. 13, the percentage of KIR positive cells increased gradually from 10% in the untreated parental cells to almost 90% in the resistant cells. This study convincingly demonstrated that KIR expression can be induced in H2122 parental cells when exposed to NK cells in vitro. This further demonstrated that cancer cells resistant to NK cell killing have acquired high levels of KIR expression on their surface.

### Example 8

### Immunohistochemistry (IHC) of KIR on Human Patient Cancer Cells

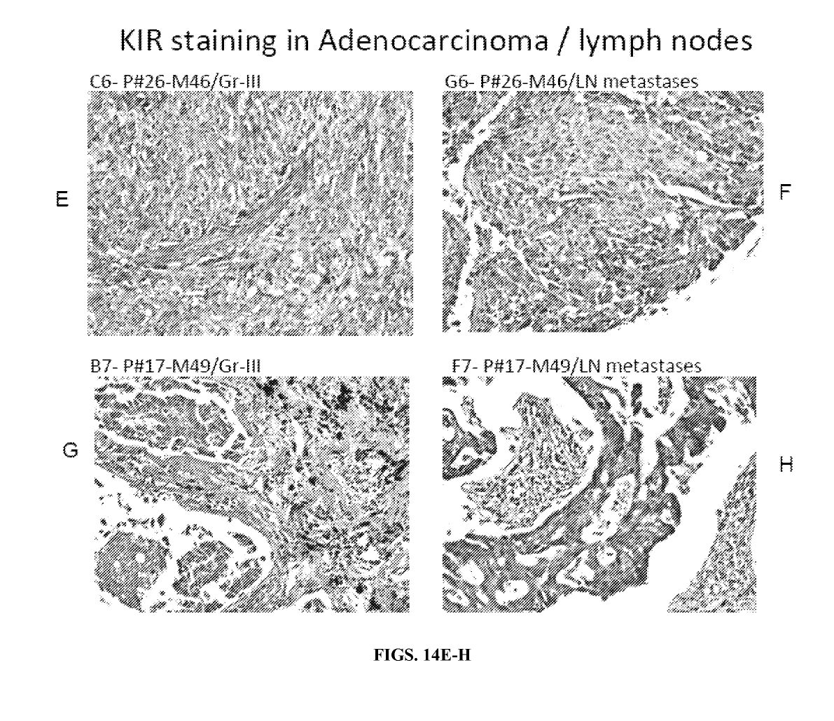

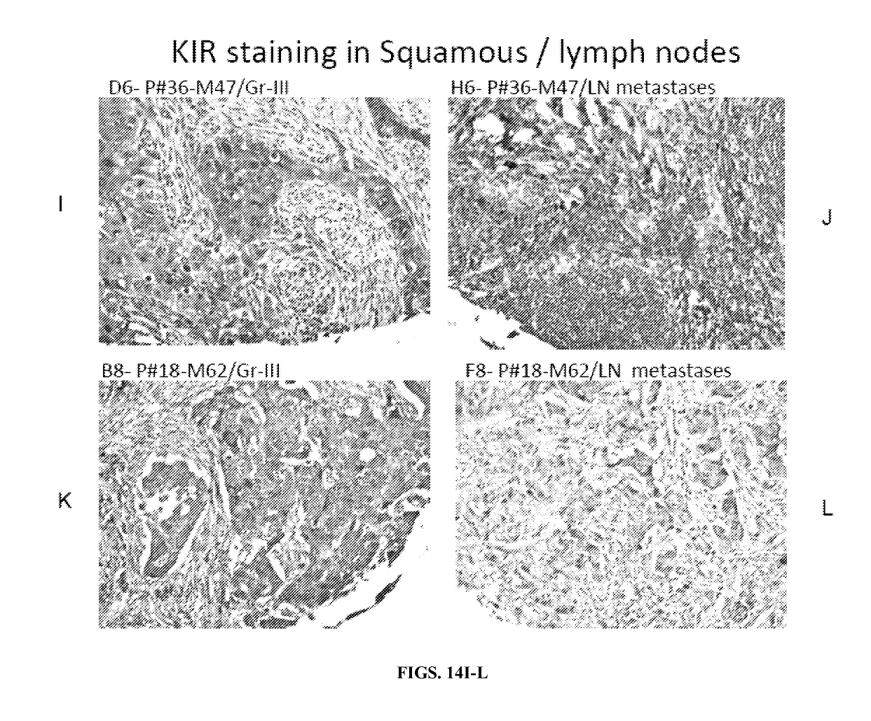

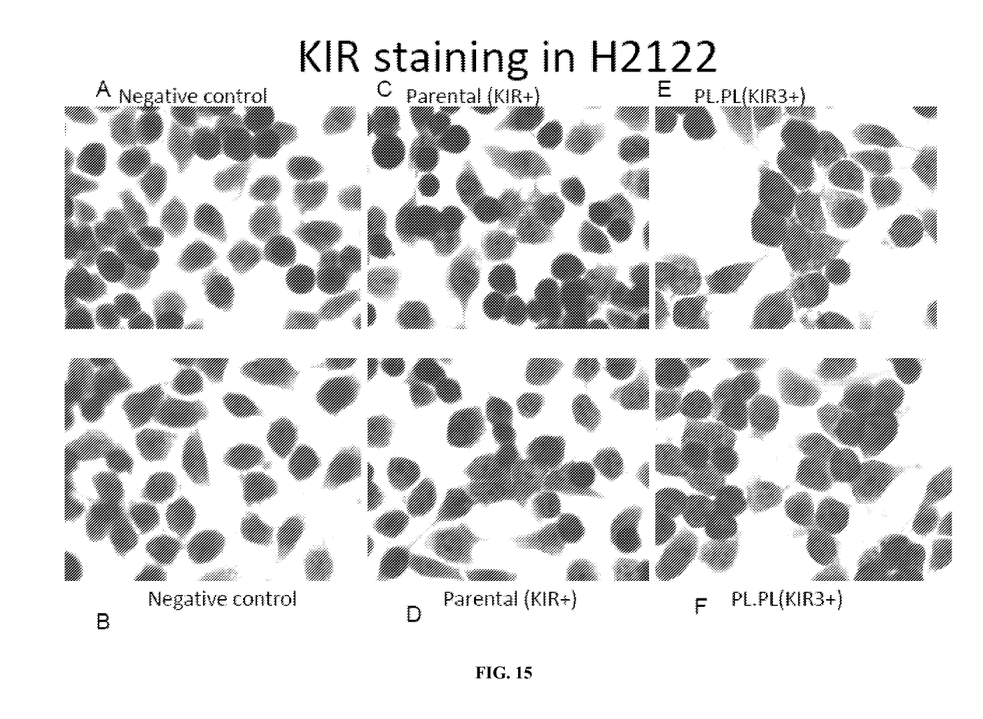

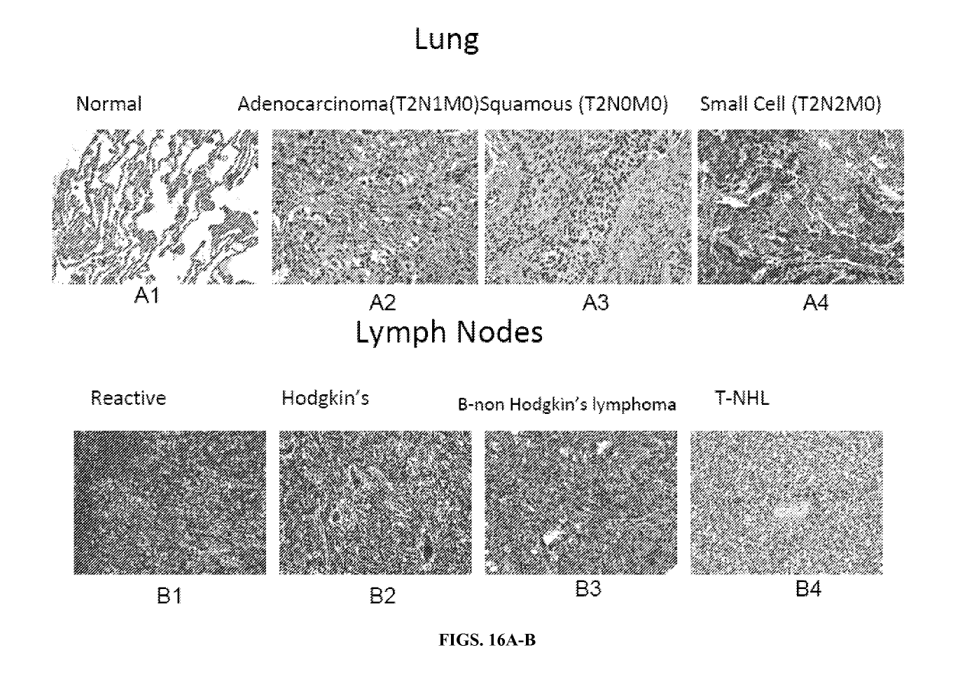

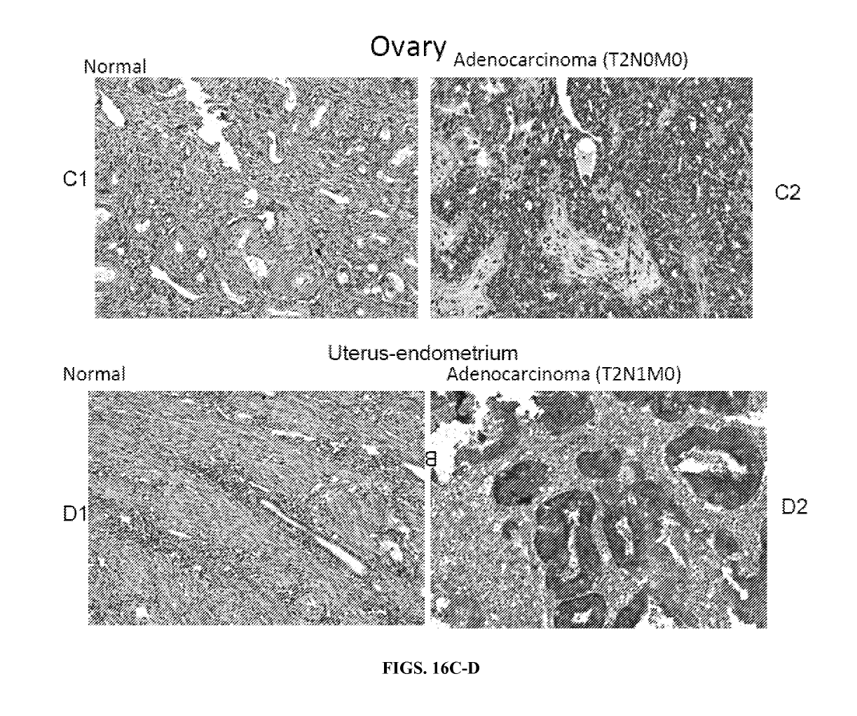

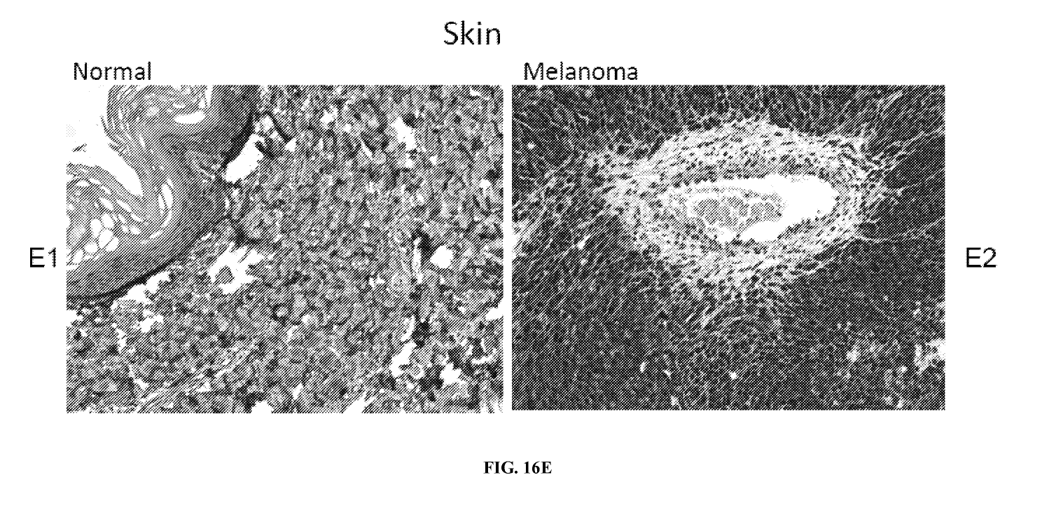

Commercial tissue micro-arrays (TMA) for human lung cancers with matched lymph node metastases, and TMA consisting of multiple human tumor types were analyzed with IHC using anti-KIR2DL1 antibodies and countered stained with H & E. H & E staining showed that KIR positive cells are cancer cells and not lymphocytes or NK cells that have infiltrated the tumor tissues. KIR immuno-reactivities are detected on the membrane as well as in the cytoplasm of the cancer cells. Interestingly, various degrees of positive KIR stainings were detected in a majority of human lung cancer cells and their matched lymph node metastases. Six matched pairs of primary tumors and lymph node specimens from two small cell lung carcinomas (SCLC), two adenocarcinomas and two squamous cell carcinomas are shown in FIG. 14A-L. KIR staining intensity in SCLC was slightly stronger than those in adenocarcinoma and squamous cells. While most of the primary tumor cells examined are of advanced stages (grade II and above) with strong KIR staining, a statistically significant increase in KIR staining intensity was not seen when compared with their matched lymph node metastases, which may be due to limited sample numbers. When immunocytochemistry was carried out directly on H2122 parental and metastatic cells grown on tissue culture chamber slides, strong membrane bound KIR staining signal was detected in most KIR positive cells with a weaker and diffusive staining in the cytoplasm, as shown in FIG. 15A-F. Strong KIR staining was detected at the junctions between KIR positive cells, indicating that KIR staining is mostly extra-cellular and membrane bound on the cancer cells, as anticipated for a transmembrane receptor. Furthermore, strong KIR staining signals were also detected in the TMA consisting of multiple human malignant tumors, such as brain, Hodgkin's lymphoma, lung, melanoma, nasopharynx, ovarian, salivary gland, uterus-cervix and endometrium tumor tissues, and their intensities are much stronger than those found in their matched normal tissues, as shown in FIG. 16A-E. Together, the IHC results showed that KIR proteins are clearly detectable on many aggressive and advanced stage cancer cells found in multiple human tumor tissues, suggesting that aberrant expression of KIR on cancer cells can be used as a strong indicator for propensity to metastasis and is likely to be a good biomarker for cancer diagnosis and prognosis.

### Example 9

### Methods

In vitro cell culture and tagging with GFP: The non-small cell lung cancer cell (NSCLC) lines A549, H157 and H2122 were obtained from the ATCC (Rockville, Md.). McF7 was obtained from Dr. Kate Horwitz, University of Colorado Denver, Mia-PaCa was obtained from Dr. David Ross, School of Pharmaceutical Sciences, University of Colorado Denver. These cell lines were maintained in RPMI-1640 media (Hyclone, Logan, Utah) supplemented with 10% fetal bovine serum (Hyclone, Logan, Utah) at 37° C. in a 5% CO₂ incubator with 100% humidity. H2122 cells were tagged with a GFP reporter construct (pLNCX2 rectroviral vector) and stable high GFP-expressing cells were selected by three rounds of flow cytometric cell sorting.

Orthotopic nude rat model and DNA microarray analysis: Six to seven week old female athymic nude rats, obtained from the National Cancer Institute (Frederick, Md.), were maintained in pathogen-limited conditions with an IACUC approved protocol at the CCM, University of Colorado Denver, Aurora, Colo. These nude rats have fully functional NK cells and B cells but are partially athymic with small remenent thymus (Jong et al., 1980; Rolstad, 2001). They were irradiated with 450 rad X-rays 24 hours prior to tumor implantation to improve tumor uptake (Howard et al., 1991). H2122-GFP single cell suspension at 1×10⁷ in 100 μl RPMI were instilled intratracheally into the left lung of isoflurane-anesthetized rats by administration through a special 3-inch 22-guage catheter (Popper & Sons, Inc., New Hyde Park, N.Y.), as described (Chan et al., 2002; Bren-Mattison et al., 2005; Kusy et al., 2005). Rats were monitored closely for changes typical of early pulmonary metastases including labored breathing. Rats bearing metastases were euthanized with an overdose of pentobarbital and dissected under UV illumination for GFP detection (Illumatool 9900, Lightool Research). GFP-tagged tumors were isolated aseptically from the primary (PL) site of implantation (left lung), from contra-lateral right lung metastases (ML), and from distant metastases (DM) if detected, including those in thymus, thoracic wall, pancreas, adrenal glands, kidneys, spleen, liver, and various lymph nodes in the abdominal cavity. These freshly dissected GFP-tagged tumor cells were mechanically disaggregated and regrown in media supplemented with antibiotics. Stocks of re-established cell lines were frozen and the rest were used within four to six weeks. Total RNAs were isolated from early passages of these GFP-tagged cells with Qiagen RNeasy kits (Qiagen, Valencia, Calif.). Samples were in vitro transcribed, labeled and hybridized to Affymetrix HG-U133 plus 2 chips (Santa Clara, Calif.) with 54,000 probe sets, and scanned on an Agilent Bioanalyzer 2100 (Agilent Technologies, Palo Alto, Calif.) at the University of Colorado Cancer Center, Microarray Core. Gene expression was analyzed with GeneSpring GX 7.31 (Agilent Technologies, Palo Alto, Calif.).

Immunofluorescence staining of KIR subtypes by flow cytometry: KIR expression in GFP-tagged tumor cells was analyzed with immunoflourescent flow cytometry using APC-conjugated primary antibodies against various KIR subtypes including CD158a (KIR2DL1), CD158b (KIR2DL2), CD158e (KIR3DL1), CD158i (KIR2DS4) and pan-KIR2D (Miltenyi Biotec, Auburn, Calif.). One million cells from each group were reacted with anti-KIR antibodies according to the manufacturer's instructions. Positive cells were analyzed with a DAKO flow Cytometer at the University of Colorado Cancer Center Flow Core. Double-positive cells (GFP and APC) with strong KIR expression intensity were sorted and enriched with FACS.

Immunohistochemistry of KIR: Commercial human tissue microarray (TMA) slides LC810 (primary lung cancers with matched metastatic lymph nodes) and MTU951 (multiple tumors) were purchased from US Biomax (Rockville, Md.). Orthotopic human lung tumors were isolated from nude rats, fixed in 10% buffered Formalin and paraffin embedded. TMAs and fixed tissue sections were deparaffinized, rehydrated, and processed with standard antigen retrieval solution in a decloaking chamber (Biocare Medical, CA) first at 120° C. for 30 sec and then at 85° C. for 10 sec. Slides were incubated with various blocking solutions (Dako S2003) for endogenous peroxidases and Dako X0590 for endogenous biotin) and reacted with primary anti-KIR2DL1 antibody conjugated with biotin (CD158a/h from Miltenyi Biotec) at 1:50 dilution overnight at 4° C. followed by a secondary biotinlyated goat anti-Mouse IgG at 1:200 dilution (Vector Laboratories: BA-9200). Proteins were visualized and developed with 3,3′-diaminobenzidine (DAKO #K3468). Mouse IgG1 (Dako X0931) was used as negative control. Specimens were counterstained with hematoxylin (Anatech #842). Samples were examined with an Axioscope (Carl Zeiss) and images were acquired with the Spot RT Camera and Software v4.0 (Diagnostic Instruments, Sterling Heights, Mich.).

NK cell isolation and cytolytic assays: Human NK cells were isolated from peripheral blood of normal humans after partial purification from Ficoll-Hypaque gradients by Miltenyi Magnetic Beads (NK cell isolation kit, #130-092-657, Miltenyi Biotech, CA) and rodent NK cells were isolated from spleens and bone marrows of homozygous nude rats (Howard et al., 1991). GFP-tagged H2122 cells from the parental cell line and a metastatic subclone (K3) with high KIR expression were incubated with human or rodent NK cells at various effector/target ratios (as indicated) in 96-well plates. Anti-growth effects of NK cells on H2122 were followed for several days using GFP fluorescent intensity measured on a BioTek Fluorescent Microplate Reader (Winooski, Vt.). After continuous co-culturing with NK cells for 14 to 21 days, surviving GFP-tagged lung cancer cells were selected and analyzed for KIR expression as described above.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,554,101
U.S. Pat. No. 4,683,195
U.S. Pat. No. 4,683,202
U.S. Pat. No. 4,684,611
U.S. Pat. No. 4,800,159
U.S. Pat. No. 4,879,236
U.S. Pat. No. 4,883,750
U.S. Pat. No. 4,952,500
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,302,523
U.S. Pat. No. 5,322,783
U.S. Pat. No. 5,324,633
U.S. Pat. No. 5,384,253
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,464,765
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,538,877
U.S. Pat. No. 5,538,880
U.S. Pat. No. 5,545,531
U.S. Pat. No. 5,547,839
U.S. Pat. No. 5,550,318
U.S. Pat. No. 5,554,501
U.S. Pat. No. 5,556,752
U.S. Pat. No. 5,561,071
U.S. Pat. No. 5,563,055
U.S. Pat. No. 5,571,639
U.S. Pat. No. 5,580,726
U.S. Pat. No. 5,580,732
U.S. Pat. No. 5,580,859
U.S. Pat. No. 5,589,466
U.S. Pat. No. 5,591,616
U.S. Pat. No. 5,593,839
U.S. Pat. No. 5,599,672
U.S. Pat. No. 5,599,695
U.S. Pat. No. 5,610,042
U.S. Pat. No. 5,610;287
U.S. Pat. No. 5,624,711
U.S. Pat. No. 5,631,134
U.S. Pat. No. 5,639,603
U.S. Pat. No. 5,654,413
U.S. Pat. No. 5,656,610
U.S. Pat. No. 5,658,734
U.S. Pat. No. 5,661,028
U.S. Pat. No. 5,665,547
U.S. Pat. No. 5,667,972
U.S. Pat. No. 5,693,762
U.S. Pat. No. 5,695,940
U.S. Pat. No. 5,700,637
U.S. Pat. No. 5,702,932
U.S. Pat. No. 5,736,524
U.S. Pat. No. 5,739,169
U.S. Pat. No. 5,744,305
U.S. Pat. No. 5,780,448
U.S. Pat. No. 5,789,215
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,155
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,981,200
U.S. Pat. No. 5,981,274
U.S. Pat. No. 5,994,624
U.S. Pat. No. 6,004,755
U.S. Pat. No. 6,020,192
U.S. Pat. No. 6,054,297
U.S. Pat. No. 6,087,102
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Patent Appln. 2008/0009439
Abbondanzo et al., Breast Cancer Res. Treat., 16:182(151), 1990.
Albini et al., Cancer Metastasis Rev., 27:75-83, 2008.
Allred et al., Breast Cancer Res. Treat., 16:182(149), 1990.
Arap et al., Cancer Res., 55(6):1351-1354, 1995.
Austin-Ward and Villaseca, Revista Medica de Chile, 126(7):838-845, 1998.
Ausubel et al., Current Protocols in Molecular Biology, Greene Publ. Assoc. Inc. & John Wiley & Sons, Inc., MA, 1996.
Azogui et al., J. Invest. Dermatol., 97:425-429, 1991.
Baichwal and Sugden, In: Gene Transfer, Kucherlapati (Ed.), Plenum Press, NY, 117-148, 1986.
Bakhshi et al., Cell, 41(3):899-906, 1985.
Barany and Merrifield, In: The Peptides, Gross and Meienhofer (Eds.), Academic Press, NY, 1-284, 1979.
Belisle et al., Immunology, 122:418-429, 2007.
Bellus, J. Macromol. Sci. Pure Appl. Chem., A31(1): 1355-1376, 1994.
Bren-Mattison et al., Oncogene, 24:1412-1422, 2005.
Brown et al., Immunol Ser, 53:69-82, 1990.
Bukowski et al., Clinical Cancer Res., 4(10):2337-2347, 1998.
Caldas et al., Cancer Res., 54:3568-3573, 1994.
Capaldi et al., Biochem. Biophys. Res. Comm., 74(2):425-433, 1977.
Carbonelli et al., FEMS Microbiol. Lett., 177(1):75-82, 1999.
Carrega et al., Cancer, 112(4):863-875, 2008
Chan et al., Clin. Cancer Res., 8:904-912, 2002.
Chandler et al., Proc. Natl. Acad. Sci. USA, 94(8):3596-601, 1997.
Chen and Okayama, Mol. Cell Biol., 7(8):2745-2752, 1987.
Cheng et al., Cancer Res., 54(21):5547-5551, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Coca et al., Cancer, 79:2320-2328, 1997.
Cocea, Biotechniques, 23(5):814-816, 1997.
Cole et al., Science, 258:1650-1654, 1992.
Coupar et al., Gene, 68:1-10, 1988.
Davidson et al., J. Immunother., 21(5):389-398, 1998.
De Jager et al., Semin. Nucl. Med., 23(2):165-179, 1993.
Doolittle and Ben-Zeev, Methods Mol, Biol, 109:215-237, 1999.
Dukers et al., J. Clin. Pathol., 54:224-228, 2001.
Dunn et al., Nature Immunology, 3:991-998, 2002
Ep Appln. 329 822
EP Appln. 373 203
EP Appln. 785 280
EP Appln. 799 897
European Application No. 320 308
Fechheimer et al., Proc Natl. Acad. Sci. USA, 84:8463-8467, 1987.
Fidler, Nat. New Biol., 242:148-149, 1973.
Fodor et al., Biochemistry, 30(33):8102-8108, 1991.
Fraley et al., Proc. Natl. Acad. Sci. USA, 76:3348-3352, 1979.
Friedmann, Science, 244:1275-1281, 1989.
Frohman, In: PCR Protocols: A Guide To Methods And Applications, Academic Press, N.Y., 1990.
GB Appln. 2 202 328
Georgiannos et al., Surgery, 134:827-834, 2003.
Gonzalez et al., Arch. Otolaryngol. Head Neck Surg., 124:852-856, 1998.
Gopal, Mol. Cell Biol., 5:1188-1190, 1985.
Gottesman and Pastan, Annu. Rev. Biochem., 62:385-427, 1993.
Graham and Van Der Eb, Virology, 52:456-467, 1973.
Gulbis and Galand, Hum. Pathol., 24(12):1271-1285, 1993.
Hacia et al., Nature Genet., 14:441-449, 1996.
Hanibuchi et al., Int. J. Cancer, 78(4):480-485, 1998.
Harland and Weintraub, J. Cell Biol., 101(3):1094-1099, 1985.
Harlow and Lane, In: Antibodies: A Laboratory Manual, Cold Spring Harbor Laboratory, Cold Spring Harbor, N.Y., 346-348, 1988.
Hellstrand et al., Acta Oncologica, 37(4):347-353, 1998.
Hermonat and Muzycska, Proc. Natl. Acad. Sci. USA, 81:6466-6470, 1984.
Horwich et al. J. Virol., 64:642-650, 1990.
Howard et al., Cancer Res., 51:3274-3280, 1991.
Hsia et al., Chang Gung Med. J., 28:335-340, 2005.
Hui and Hashimoto, Infection Immun., 66(11):5329-5336, 1998.
Hussussian et al., Nat. Genet., 8(1):15-21, 1994.
Innis et al., Proc. Natl. Acad. Sci. USA, 85(24):9436-9440, 1988.
Inouye and Inouye, Nucleic Acids Res., 13:3101-3109, 1985.
Ishigami et al., Cancer, 88:577-583, 2000.
Johnson et al., In: Biotechnology And Pharmacy, Pezzuto et al. (Eds.), Chapman and Hall, NY, 1993.
Jong et al., Clin. Immunol. Immunopathol., 17:163-172, 1980.
Ju et al., Gene Ther., 7(19):1672-1679, 2000.
Kaeppler et al., Plant Cell Reports, 9:415-418, 1990.
Kamb et al., Nat. Genet., 8(1):23-26, 1994.
Kamb et al., Science, 2674:436-440, 1994.
Kaneda et al., Science, 243:375-378, 1989.
Kato et al, J. Biol. Chem., 266:3361-3364, 1991.
Kerr et al., Br. J. Cancer, 26(4):239-257, 1972.
Kopfstein and Christofori, Cell Mol. Life Sci. 63:449-468, 2006.
Kusy et al., Neoplasia, 7:457-465, 2005.
Kwoh et al., Proc. Natl. Acad. Sci. USA, 86:1173, 1989.
Kyte and Doolittle, J. Mol. Biol., 157(1):105-132, 1982.
Levenson et al., Hum. Gene Ther., 9(8):1233-1236, 1998.
Lorusso and Ruegg, Histochem. Cell. Biol., 130:1091-1103, 2008.
MacBeath and Schreiber, Science, 289(5485):1760-1763, 2000.
Macejak and Sarnow, Nature, 353:90-94, 1991.
Mealey et al., Cancer Lett., 126:187-192, 1998.
Merrifield, Science, 232(4748):341-347, 1986.
Mitchell et al., Ann. NY Acad. Sci., 690:153-166, 1993.
Mitchell et al., J. Clin. Oncol., 8(5):856-869, 1990.
Mori et al., Cancer Res., 54(13):3396-3397, 1994.
Morton et al., Arch. Surg., 127:392-399, 1992.
Nakamura et al., In: Handbook of Experimental Immunology (4^{th} Ed.), Weir et al., (eds). 1:27, Blackwell Scientific Publ., Oxford, 1987.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Nobori et al., Nature (London), 368:753-756, 1995.
Ohara et al., Proc. Natl. Acad. Sci. USA, 86:5673-5677, 1989.
Okamoto et al., Proc. Natl. Acad. Sci. USA, 91(23):11045-11049, 1994.
Omirulleh et al., Plant Mol. Biol., 21(3):415-428, 1993.
Orlow et al., Cancer Res, 54(11):2848-2851, 1994.
Orlow et al., Int. J. Oncol., 15(1):17-24, 1994.
Ostrand-Rosenberg, Curr. Opin. Genet. Dev., 18:11-18, 2008.
Pandey and Mann, Nature, 405(6788):837-846, 2000.
Parham, Nat. Rev. Immunol., 5:201-214, 2005.
PCT Appln. PCT/US87/00880
PCT Appln. PCT/US89/01025
PCT Appln. WO 013/8580
PCT Appln. WO 016/8255
PCT Appln. WO 030/20898
PCT Appln. WO 030/22421
PCT Appln. WO 030/23058
PCT Appln. WO 030/29485
PCT Appln. WO 030/40410
PCT Appln. WO 030/53586
PCT Appln. WO 030/66906
PCT Appln. WO 030/67217
PCT Appln. WO 030/76928
PCT Appln. WO 030/87297
PCT Appln. WO 030/91426
PCT Appln. WO 030/93810
PCT Appln. WO 031/00012
PCT Appln. WO 031/00448A1
PCT Appln. WO 04/048933
PCT Appln. WO 040/20085
PCT Appln. WO 040/27093
PCT Appln. WO 88/10315
PCT Appln. WO 89/06700
PCT Appln. WO 90/07641
PCT Appln. WO 93/17126
PCT Appln. WO 94/09699
PCT Appln. WO 95/06128
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 99/23256
PCT Appln. WO 99/35505
PCT Appln. WO 99/36760
Pease et al., Proc. Natl. Acad. Sci. USA, 91:5022-5026, 1994.
Pelletier and Sonenberg, Nature, 334(6180):320-325, 1988.
Pietras et al., Oncogene, 17(17):2235-2249, 1998.
Poste et al., Cancer Res., 40:1636-1644, 1980.
Potrykus et al., Mol. Gen. Genet., 199(2):169-177, 1985.
Potrykus et al., Mol. Gen. Genet., 199:183-188, 1985.
Qin et al., Proc. Natl. Acad. Sci. USA, 95(24):14411-14416, 1998.
Rajaram et al., Br. J. Cancer, 62(2):205-208, 1990.
Ravindranath and Morton, Intern. Rev. Immunol., 7: 303-329, 1991.
Reiman et al., Semin. Cancer Biol., 17:275-287, 2007.
Ridgeway, In: Vectors: A Survey of Molecular Cloning Vectors and Their Uses, Rodriguez et al. (Eds.), Stoneham: Butterworth, 467-492, 1988.
Rippe, et al., Mol. Cell Biol., 10:689-695, 1990.
Rolstad, Immunol. Rev., 184:136-144, 2001.
Rosenberg et al., Ann. Surg. 210(4):474-548, 1989.
Rosenberg et al., N. Engl. J. Med., 319:1676, 1988.
Sambrook et al., In: Molecular cloning, Cold Spring Harbor Laboratory Press, Cold Spring Harbor, N.Y., 2001.
Sambrook et al., In: Molecular cloning: a laboratory manual, 2^{nd} Ed., Cold Spring Harbor Laboratory Press, Cold Spring Harbor, N.Y., 1989.
Schleypen et al., Clin. Cancer Res., 12:718-725, 2006.
Serrano et al., Nature, 366:704-707, 1993.
Serrano et al., Science, 267(5195):249-252, 1995.
Shoemaker et al., Nature Genetics, 14:450-456, 1996.
Smyth et al., Adv. Immunol., 90:1-50, 2006.
Stewart and Young, In: Solid Phase Peptide Synthesis, 2d. ed., Pierce Chemical Co., 1984.
Swann and Smyth, J. Clin. Invest., 117:1137-1146, 2007.
Taketomi et al., Cancer, 83:58-63, 1998.
Tam et al., J. Am. Chem. Soc., 105:6442, 1983.
Tarle et al., Urol. Res., 21:17-21, 1993.
Temin, In: Gene Transfer, Kucherlapati (Ed.), NY, Plenum Press, 149-188, 1986.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
Uhrberg et al., Immunity, 7:753-763, 1997.
UK Appln. 8 803 000
Vaguer et al., Gynecol. Oncol., 36:90-92, 1990.
Waldhauer and Steinle, Oncogene, 27:5932-5943, 2008.
Walker et al., Proc. Natl. Acad. Sci. USA, 89:392-396, 1992.
Wong et al., Gene, 10:87-94, 1980.
Yang et al., Proc. Natl. Acad. Sci. USA, 98:2616-2621, 2001.
Zitvogel et al., Nat. Rev. Immunol., 6:715-727, 2006.

1. A method of evaluating cancer in a patient comprising determining the level of KIR in a biological sample containing cancer cells obtained from the patient, wherein an elevated level of KIR in the cancer cells as compared to a control is indicative of an aggressive metastatic form of cancer and/or a poor prognosis.

2. The method of claim 1, further comprising assessing KIR level in a control sample.

3. The method of claim 1, wherein KIR mRNA level of synthesis is evaluated.

4. The method of claim 1, wherein KIR protein level of synthesis is evaluated.

5. The method of claim 1, wherein the sample is a biopsy from a tumor.

6. The method of claim 5, wherein the biopsy is from a metastasized tumor, a lymph node, or bone marrow.

7.-8. (canceled)

9. The method of claim 1, wherein the sample is a serum sample, a pleural fluid sample, a peritoneal fluid sample, a spinal fluid sample, a bronchoalveolar lavage fluid sample, a cerebral spinal fluid sample, a pleural effusion sample, or a peritoneal effusion sample.

10. (canceled)

11. The method of claim 1, wherein the control is a sample from non-cancerous tissue.

12. The method of claim 1, wherein the control is a sample from non-metastasized cancerous tissue.

13. The method of claim 1, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

14.-16. (canceled)

17. The method of claim 1, wherein an elevated level of KIR as compared to the control is indicative of an aggressive metastatic form of cancer or a poor prognosis.

18. (canceled)

19. The method of claim 1, further comprising treating the cancer based on the level of KIR.

20. (canceled)

21. The method of claim 1, wherein the patient exhibits symptoms of cancer, is at risk for cancer, or has been diagnosed with cancer.

22. The method of claim 1, further comprising obtaining a patient sample.

23. The method of claim 1, further comprising reporting the level of KIR on a cancer cell in the sample.

24. The method of claim 23, further comprising reporting the level of KIR on a cancer cell in the control.

25. The method of claim 1, wherein KIR on a cancer cell is evaluated on an array or biochip.

26. A method for evaluating cancer in a patient comprising:
(a) obtaining a biological sample containing cancer cells from the patient;
(b) obtaining information regarding the level of KIR expression in the cancer cells in the sample, wherein an elevated level of KIR on the cancer cells as compared to a control is indicative of an aggressive metastatic form of cancer and/or a poor prognosis.

27. A method of treating a cancer tumor comprising administering to a cancer patient an effective amount of a KIR inhibitor.

28. (canceled)

29. The method of claim 27, wherein the KIR inhibitor is a nucleic acid, siRNA, a peptide, an antibody, or a small molecule.

30.-36. (canceled)

37. A method of monitoring treatment of cancer in a patient comprising:
(a) determining the level of KIR in a first sample from the patient;
(b) determining the level of KIR in a second sample from the patient after treatment is effected; and
(c) comparing the level of KIR in the first sample with the level of KIR in the second sample to assess a change and monitor treatment.

38. (canceled)

39. A method of screening for a KIR inhibitor comprising
(a) contacting KIR with a candidate substance; and
(b) assaying the level of KIR, wherein a reduction in KIR indicates that the candidate substance is a candidate KIR inhibitor.

40. The method of claim 39, wherein the candidate inhibitor is a nucleic acid, siRNA, a protein, or a small molecule.

41.-43. (canceled)
